(19) United States
(12) Patent Application Publication
(10) Pub. No.: US 2026/0265161 A1
(43) Pub. Date: Sep. 10, 2026

(54) PROCESS AND PLANT FOR THE CONVERSION OF OXYGENATES TO C5+ HYDROCARBONS BOILING IN THE JET FUEL BOILING RANGE

(71) Applicant: TOPSOE A/S, Kgs. Lyngby (DK)

(72) Inventors: Finn JOENSEN, Kgs. Lyngby (DK); Martin Dan Palis SØRENSEN, Kgs. Lyngby (DK); Mads Kristian KAARSHOLM, Kgs. Lyngby (DK); Pablo BEATO, Kgs. Lyngby (DK); Angelica HIDALGO VIVAS, Kgs. Lyngby (DK); Jeremy Neil BURN, Kgs. Lyngby (DK)

(73) Assignee: TOPSOE A/S, Kgs. Lyngby (DK)

(21) Appl. No.: 19/165,489

(22) PCT Filed: Mar. 26, 2024
(86) PCT No.: PCT/EP2024/058096

§ 371 Date: Sep. 15, 2025

(30) Foreign Application Priority Data
Mar. 29, 2023 (DK) PA202300285

(51) Int. Cl. C07C 1/24; B01J 8/02; B01J 29/70; C07C 2/04; C07C 7/09

(52) U.S. Cl. CPC C07C 1/24; B01J 8/0278; B01J 8/0285; B01J 29/703; C07C 2/04; C07C 7/09; B01J 2208/00035; B01J 2208/00176

(57) ABSTRACT

Process and plant for the conversion of oxygenates to hydrocarbons boiling in the jet fuel boiling range, comprising the steps of continuously: a) providing one or more feed streams of one or more oxygenate compounds; b) heating the one or more feed streams to an inlet temperature of a first set (R1) of one or more downstream adiabatic fixed bed reaction zones; c) introducing the one or more heated feed streams (1) into inlet of R1; d) converting in the one or more adiabatic fixed bed reaction zones of R1 the one or more heated feed streams in the presence of a conversion catalyst comprising a zeolite with a framework having a 10-ring pore structure, said 10-ring pore structure being a unidimensional (1D) pore structure, to a converted-oxygenate product comprising C5+ hydrocarbons of which at least 30 wt % are said hydrocarbons boiling in the jet fuel boiling range; wherein said 1-D pore structure is any of *MRE (ZSM-48), MTT (ZSM-23), TON (ZSM-22), or combinations thereof, the content of phosphorous (P) in the conversion catalyst is 0.1-3 wt %; e) withdrawing from the one or more adiabatic fixed bed reaction zones the converted-oxygenate product; f) determining at outlet of the one or more a diabatic fixed bed reaction zones an amount of one or more unconverted oxygenate com-pounds in the withdrawn converted-oxygenate product; g) continuously adjusting the in-let temperature of the one or more feed streams in step b to maintain a constant amount of the one or more unconverted-oxygenate compounds as determined in step f of between 10 and 4000 ppmv.

### FIELD OF THE INVENTION

The present invention relates to an improved process and plant (system) for producing distillate boiling range hydrocarbons, such as naphtha, diesel and jet fuel, for instance jet fuel as sustainable aviation fuel (SAF).

### BACKGROUND

To limit the continued increase of global heating and consequently increasing world temperatures, actions must be taken to reduce human consumption of fossil fuels. Cost re-ductions of renewable energy enables decarbonization of certain specific applications, however aviation is one of the sectors where direct electrification is not feasible. What is needed are methods that efficiently convert carbon neutral feed into distillate boiling range hydrocarbons, here in particular jet fuels.

U.S. Pat. No. 8,198,338 describes a process for producing gasoline from carbon dioxide and water. C4-hydrocarbons and other hydrocarbons not suitable for high octane fuel production are recycled by partial oxidation, steam reforming, autothermal reforming or by complete combustion.

U.S. Ser. No. 11/130,718 describes an integrated process where distillate boiling range hydrocarbons are produced from methane via reforming. The reformed product is converted to methanol, the methanol is converted in first reactor to an effluent comprising olefins, and the effluent comprising olefins is together with a recycled naphtha stream converted a downstream reactor arranged in series to an oligomerized effluent. Thus, this citation discloses a “two-reactor”, or analogously, “two-steps” type system for producing the distillates from methanol.

U.S. Pat. No. 4,482,772 describes an integrated process for converting methanol or the like to heavy hydrocarbon products, especially distillate range hydrocarbons. A “two-stage” or analogously a “two-reactor” process is provided, in which the first stage is the conversion of oxygenate feedstock to lower olefins and gasoline. By-product aromatics are passed through a second stage, oligomerization. Distillate range hydrocarbons are recovered downstream.

US 2018/0155637 describes a “two-reactor” process for integration of oxygenate conversion with olefin oligomerization. The integrated process produces gasoline of a desired octane and/or distillate fuel of a desired cetane.

US 2017/0121237 also describes a “two-reactor” type system where the reactors are arranged in series and whereby a feed comprising oxygenate, e.g. methanol, dimethyl ether (DME), mixtures thereof, etc. is converted to gasoline boiling range components and distillate boiling range components, optionally without the need of significant compression between methanol conversion (at pressure P1) and subsequent oligomerization step (at pressure P2).

Similarly, WO 2010/097175 A1 discloses a process for conversion of oxygenates to liquid hydrocarbons in which a first and second reactor are also located in series, but with no compression between the reactors. In addition, the first reactor is operated at high temperatures to provide an intermediate product comprising light olefins of C2-C4, while the second reactor is operated at lower temperature to provide a mixture of liquid hydrocarbons including heavy olefins of C5.

Applicant's WO 2022063992 discloses oxygenate conversion (methanol to olefins, MTO) conducted with ZSM-48 in the temperature range 320-480° C.

It is known to prepare a phosphorous (P) modified zeolite containing catalyst, in which the zeolite is ZSM-5, and where the preparation of the P-modified zeolite is by impregnation of zeolite plus binder with a P-containing compound.

WO 2011089262 relates to the use of a catalyst in a MTO process to convert an alcohol or an ether into light olefins wherein said catalyst comprises a phosphorus modified zeolite.

The prior art is thus at least silent on actively regulating the oxygenate slip, e.g. methanol slip, at outlet the oxygenate conversion reactor.

Hence, conventionally the conversion of methanol or other oxygenates into distillate boiling range hydrocarbons is conducted in two steps and with no active regulation of the oxygenate slip to control the process. First, the oxygenates are converted into light (short) olefins, typically ethylene, propylene, and butylene, and some other light or intermediate olefins (with carbon number<C9). This first step is conducted at low reactant partial pressures, low residence times (high space velocities), and high temperature, e.g. above 370° C., to facilitate the production of short olefins i.e. olefins with low carbon numbers. In the second step, the stream containing such intermediate olefins is pressurized, before it is fed into a second reactor system operating at an elevated pressure and lower temperature to facilitate carbon chain growth. The second reactor is typically an oligomerization reactor (OLI reactor) where the olefins produced upstream in a methanol to olefins reactor (MTO reactor) are converted into C8-C19 such C8-C17 or C8-C16 hydrocarbons, which correspond to the jet fuel boiling range. A subsequent hydroprocessing step is typically also conducted. Further, in between the MTO reactor and the OLI reactor, as for instance disclosed in above-cited US 2018/0155637 A1, at least water is normally removed as it is may be detrimental for the downstream oligomerization.

Hence, at least two sequential steps are required to produce the hydrocarbons in the jet fuel boiling range.

It would therefore be desirable to provide a superior process and plant for producing hydrocarbons boiling in the jet fuel range, i.e. a simpler process which in a single step of oxygenate conversion and thus more directly is capable of producing a high proportion of said hydrocarbons.

Applicant's U.S. Ser. No. 11/060,036 (corresponding to US2021002557 or WO2019219397) describes an improved process for the conversion of oxygenates to C5+ hydrocarbons boiling in the gasoline boiling range, in which the inlet temperature of 350° C. of a feed stream to an oxygenate conversion reactor provided with, implicitly the well-known ZSM-5 zeolite as catalyst for oxygenate conversion to gasoline in a fixed bed, is continuously adjusted to maintain a constant amount of the unconverted oxygenate at the outlet of the reactor, as well as a constant level of conversion of oxygenate between 95% to 99.9%.

This citation proposes also operation with a lower inlet temperature to the reactor with the purpose of reducing the rate of dealumination of the ZSM-5 zeolite (which is a zeolite having a 3-dimensional (3D) 10 ring pore structure) and thereby reducing the rate of catalyst deactivation.

However, it has now been found that when such low temperatures in the fixed bed of the reactor are maintained, deactivation of catalysts comprising a zeolite having a 1D 10 ring pore structure, such as ZSM-48, may increase due an increasing impact of coking: at lower temperatures the oxygenate conversion rate is lower, which means that the impact of coking is accentuated and thus more visible on the measured oxygenate conversion, whereby the oxygenate conversion reaction will extinguish quicker. Thus, the apparent catalyst stability decreases when the temperature decreases, thereby leading to an in-stable process for producing hydrocarbons. On the other hand, operating at higher temperature also conveys catalyst deactivation due to a higher degree of dealumination of the catalyst via the so-called steaming, i.e. exposure of the catalyst to vapor water thereby dealuminating the catalyst. The catalyst comprises a zeolite having a given silica-to-alumina ratio (SAR) and is desirable to minimize dealumination of the catalyst for prolonging its longevity, i.e. lifetime. Coke (pre-graphitic and graphitic type) deposited in the catalyst at higher reaction temperatures also conveys catalyst deactivation and is therefore removed by periodical burning off, thereby regenerating the catalyst. This normally requires i.a. a high energy input to provide high oxidation temperatures and/or high oxygen environment, thereby generating CO₂ emissions. The deposited carbon, due to the higher reaction temperatures during oxygenate conversion, has a lower oxidation reactivity, thus requiring higher temperatures for its burning during regeneration of the catalyst.

Hence, while reducing the operating temperature during oxygenate conversion may at first glance appear to overcome the challenge of catalyst dealumination, coking of the catalyst at the low temperatures still occurs, thereby leading to an unstable process.

Applicant's WO 2022063992 discloses in the examples oxygenate conversion (methanol to olefins, MTO) conducted with ZSM-48 in the temperature range 320-480° C., with the proportion of C8-C17 olefins produced therein being lower than 10 wt %.

Applicant's WO2022063994 discloses the conversion of an olefin stream to hydrocarbons boiling in the jet fuel range in a combined oligomerization and hydrogenation step. In a particular embodiment, a process for oxygenate conversion to said olefin stream using a ZSM-48 zeolite, is disclosed.

It would therefore be desirable to provide a process and plant for producing distillate boiling range products such as jet fuel in a stable manner despite catalyst coking, while at the same time reducing deactivation of the oxygenate conversion catalyst due to dealumination, thus also increasing catalyst longevity.

It would also be desirable to provide a process and plant with a lower energy input during catalyst regeneration. In connection thereto, it would be desirable to provide a lower carbon footprint by reducing CO₂ emissions during catalyst regeneration by burning off coke deposited in the catalyst, via reduction of the consumption of hydrocarbon fuel utilized during the regeneration, such as when utilizing a fired heater and feeding natural gas as fuel for the burning.

Thus, more generally it would be desirable to provide a superior process and plant for producing hydrocarbons boiling in the jet fuel range which in a single step is capable of constantly, i.e. in a stable manner, producing a high proportion of said hydrocarbons, while at the same time increasing the longevity of the oxygenate conversion catalyst by reducing its rate of dealumination, as well as enabling a lower energy input and thereby lower carbon footprint during regeneration of the oxygenate conversion catalyst.

Applicant's co-pending European patent application EP22214261.4 addresses the above-mentioned problems.

Now a further improvement of the process has also been found by increasing the proportion of olefins in the C5+ fraction of converted oxygenate product, or the proportion of olefins in the converted oxygenate product, and thereby the jet fuel yield.

### SUMMARY OF THE INVENTION

The present application discloses an improved integrated process and plant for producing sustainable distillate boiling range hydrocarbons, in particular jet fuel. The conversion from oxygenates into distillate boiling range hydrocarbons is conducted in a single step utilizing surprising reaction synergies realized from experimental tests performed in a 0.6 bpd pilot plant, along with experiments conducted in laboratory reactors at particular silica to alumina ratios (SAR) and phosphorous (P) content in the catalyst, as shown in the Examples section of the present application.

In a first general embodiment according to a first aspect of the invention, the invention provides a process for the conversion of oxygenates to hydrocarbons boiling in the jet fuel boiling range, comprising the steps of continuously:
a) providing one or more feed streams of one or more oxygenate compounds;
b) heating the one or more feed streams to an inlet temperature of a first set (R1) of one or more downstream adiabatic fixed bed reaction zones;
c) introducing the one or more heated feed streams into inlet of R1, i.e. into inlet of the one or more adiabatic fixed bed reaction zones of R1;
d) converting in the one or more adiabatic fixed bed reaction zones of R1 the one or more heated feed streams in the presence of a conversion catalyst comprising a zeolite with a framework having a 10-ring pore structure, said 10-ring pore structure being a unidimensional (1D) pore structure, to a converted-oxygenate product comprising C5+ hydrocarbons of which at least 30 wt % are said hydrocarbons boiling in the jet fuel boiling range; wherein said 1-D pore structure is any of *MRE (ZSM-48), MTT (ZSM-23), TON (ZSM-22), or combinations thereof and the content of phosphorous (P) in the catalyst is 0.1-3 wt % (0.1-3 wt % P);
e) withdrawing from the one or more adiabatic fixed bed reaction zones the converted-oxygenate product;
f) determining at outlet of the one or more adiabatic fixed bed reaction zones an amount of one or more unconverted oxygenate compounds in the withdrawn con-verted-oxygenate product;
g) continuously adjusting the inlet temperature of the one or more feed streams in step b to maintain a constant amount of the one or more unconverted-oxygenate compounds as determined in step f of between 10 and 4000 ppmv; i.e. at outlet of the one or more adiabatic fixed bed reaction zones (reactor effluent).

Thereby, highly surprisingly, in a single step a high proportion of hydrocarbons boiling in the jet fuel range are produced with a catalyst comprising a zeolite with a 1-D pore structure such as ZSM-48, and which according to laboratory tests has not produced any measurable fraction of hydrocarbons boiling in the jet fuel range. Hence, there is a highly surprising chain length addition towards jet fuel hydrocarbons when moving from laboratory tests with a catalyst comprising a zeolite with a 1-D pore structure such as ZSM-48, into the oxygenate slip method with the same zeolite as in the present invention. A sizable jet fuel proportion is highly surprisingly produced in one step by using an oxygenate slip method, e.g. methanol slip method, that was previously used with a different zeolite, namely ZSM-5 which is a zeolite with 3-D, with the purpose of producing a different and lighter product, namely gasoline (typically C5-C8 hydrocarbons), as for the latter the laboratory tests of gasoline producing zeolite (ZSM-5) compared to using the oxygenate slip method with same ZSM-5 did not produce a corresponding chain length addition. The present invention is thus not simply about producing jet fuel, but about producing a sizeable proportion of jet fuel already in the converted oxygenate product, thus in one step.

In an embodiment, the zeolite has a silica-to-alumina ratio (SAR) of at least 100, such as at least 110.

In an embodiment, the zeolite has a SAR of above 130.

The SAR is based on Si and Al X-ray fluorescence (XRF) analysis, as is well-known in the art.

In an embodiment, said 1-D pore structure is ZSM-48, the pressure is at least 10 barg; the inlet temperature in step b of the one or more downstream adiabatic fixed bed reaction zones is 275° C. or higher, and the outlet temperature in step f of the one or more downstream adiabatic fixed bed reaction zones is 475° C. or lower; the SAR is at least 150, such as 150-200, and the content of P in the conversion catalyst is 0.5-1.5 wt %(0.5-1.5 wt % P), such as 0.8-1.2 wt % (0.8-1.2 wt % P).

In an embodiment, the conversion catalyst is provided as an extrudate and wherein a P-precursor, such as phosphoric acid, is impregnated in said extrudates for providing said content of P of 0.1-3 wt % P or said 0.5-1.5 wt % in the conversion catalyst; and optionally, wherein 45-65 wt %, such as 50-60 wt %, of the conversion catalyst is extruded with alumina.

In an embodiment, the one or more oxygenate compounds is methanol, dimethyl ether (DME), or combinations thereof.

Thereby, the oxygenate conversion is operated in a dynamic manner that maintains both oxygenate conversion and yields of hydrocarbons boiling in the jet fuel range stable over time, irrespectively of catalyst deactivation. By converting an oxygenate such as methanol over the conversion catalyst having a 10-ring 1D pore structure zeolite and adjusting the reactor inlet temperature to maintain a certain oxygenate (e.g. methanol) slip at outlet R1, there is a constant, i.e. stable, and high yield of hydrocarbons boiling in the jet fuel boiling range. Astonishingly, at least 30 wt % of the total C5+ hydrocarbons produced are already hydrocarbons boiling in the jet fuel boiling range, or at least 30 wt % of the converted-oxygenate product, are already hydrocarbons boiling in the jet fuel boiling range, i.e. C8+ hydrocarbons, such as C8-C17 hydrocarbons. For instance, at least 50 wt % of the total C5+ hydrocarbons produced are already C8-C17 hydrocarbons i.e. a C8-C17 hydrocarbon fraction. For instance, at least 45 wt % of the converted-oxygenate product are already C8-C17 hydrocarbons i.e. a C8-C17 hydrocarbon fraction. Furthermore, a high proportion of at least the C8+ hydrocarbons, is also already provided as olefins.

The skilled person would understand that this is a highly significant and completely unexpected deviation from producing hydrocarbons in the gasoline boiling range, the latter meaning C5-C12 hydrocarbons. In connection thereto, it would also be appreciated by the skilled person that the product pool for gasoline is very different from that of jet fuel, as is shown in appended FIG. 7 which depicts typical carbon numbers for gasoline, jet fuel and diesel.

Furthermore, it has been found that when conducting the conversion of the oxygenates at said particular combination of temperatures, zeolite framework, SAR, as well as P content in the catalyst, the olefin content in the C5+ fraction of converted oxygenate product is significantly increased; for instance 80 wt % or more of the C5+ fraction are olefins compared to where there is no P in the catalyst and/or where the SAR is lower, as shown in connection with Examples. The production of less desirable compounds such as paraffins, isoparaffins, naphthenes and aromatics, is maintained at a minimum.

In an embodiment, as recited above, the SAR is above 130, suitably at least 150, such as 150-200. For instance, the SAR is 150, 160, 170, 180, 190, 200, 210, 220, 230, 240 or 250. In another embodiment, the SAR is 100, 110, 120 or 130.

As already recited, the SAR is based on Si and Al X-ray fluorescence (XRF) analysis, as is well-known in the art.

The P content in the conversion catalyst is 0.1-3 wt %, suitably 0.8-1.2 wt %. For instance, the P content is 0.2, 0.3, 0.4, 0.5, 0.6, 0.7, 0.8, 0.9, 1.0, 1.1 or 1.2 wt %.

In an embodiment, as recited above, the conversion catalyst is provided as an extrudate; and a P-precursor, such as phosphoric acid, is impregnated in said extrudates for providing said 0.1-3 wt % P in the conversion catalyst. This represents a simple way of providing the catalyst, as this implies impregnating the catalyst after a binder has been incorporated, instead of incorporating the P earlier in the process for manufacturing the catalyst, such as by incorporating P in the zeolite prior to incorporating the binder and optional additional components. It would thus be understood that the conversion catalyst may also comprise a binder and said optional additional components such as clays. Suitably, the catalyst comprises a binder selected from the group of alumina, aluminum phosphate, silica, silica-alumina, zirconia, titania and combinations of these metal oxides, and other refractory oxides, and clays such as montmorillonite, kaolin, palygorskite, smectite and attapulgite; preferably an aluminum-based binder, such as alumina, aluminum phosphate, silica-alumina and clays. The catalyst may contain up to 30-90 wt % zeolite with the binder, suitably 50-80 wt %, the binder suitably comprising an alumina component such as a silica-alumina.

It would be understood that the content of P with respect to the weight of conversion catalyst, does not include any P already provided in the binder, such as an aluminum phosphate binder.

The process of the invention is operated on purpose with no complete conversion of oxygenates, such as with incomplete conversion of methanol as the oxygenate. Instead, the oxygenate conversion is kept at a level corresponding to the unconverted oxygenate at the outlet, herein also referred to as “oxygenate slip” or for more simplicity “methanol slip”, of 10-4000 ppmv, thereby capturing the optima for C5+ hydrocarbons yield and yield of the desired hydrocarbons boiling in the jet fuel range, such as C8-17 hydrocarbons. Yet again, it has been highly surprising, that this high amount of hydrocarbons already in the desired jet fuel boiling range could be produced under these conditions, and also completely unexpected, that a reactor operation method similar to that of applicant's U.S. Ser. No. 11/060,036 (WO 2019219397) utilized for a completely different purpose, namely for the conversion of oxygenates to C5+ hydrocarbons boiling in the gasoline boiling range under the presence of a 3D pore structure zeolite (ZSM-5, typical for gasoline production), also provides optimal conditions for production of distillate boiling range hydrocarbons, in particular the desired hydrocarbons boiling in the jet fuel boiling range, e.g. C8-17 hydrocarbons. This takes place directly from an oxygenate such as methanol, in a fixed bed reactor carrying out the oxygenate conversion, without a subsequent oligomerization step, the latter typically being required for increasing the number of carbons of the hydrocarbon product resulting from the oxygenate conversion and thus providing the hydrocarbons in the jet fuel boiling range.

Hence, providing a converted-oxygenate product comprising C5+ hydrocarbons of which already at least 30 wt % are said hydrocarbons boiling in the jet fuel boiling range, enables that a dedicated subsequent oligomerization step to produce such hydrocarbons in the jet fuel range may be obviated, or that at least the load thereto is significantly reduced e.g. throughput on a second reactor set (R2) operating as an oligomerization reactor, and which is suitably operated in parallel at least with respect to the oxygenate feed, for instance in parallel with a naphtha stream produced downstream, as recited in one or more of below embodiments and illustrated in appended FIG. 1.

The invention enables therefore also the production of distillate boiling range hydrocarbons, such as naphtha, jet fuel and diesel, in e.g. a single synthesis loop thereby providing a significant reduction in process complexity, capital expenditures and operating expenses: there is one oxygenate conversion loop instead of two loops, and thereby there is no need for an additional compression stage and no need for an additional recycle compressor; for instance where the first step is methanol to olefins (in a MTO reactor) with its associated loop for recycling streams, and the second step is oligomerization of olefins in an OLI reactor with its associated loop for recycling streams.

Yet the invention is also suitable for operation of separate loops. The provision of two separate loops is therefore also envisaged, namely an oxygenate conversion loop and an oligomerization loop. Suitably, each loop comprises its own first separation unit whereby a process condensate is separated from the hydrocarbons, as well as its own second separation unit, such as fractionation unit, for separating the hydrocarbons into the different hydrocarbon fractions, such as jet fuel and naphtha. This approach of providing separate loops is advantageous in plants of a sizable capacity, where a common fractionation unit becomes too big to accommodate the output from R1 and R2.

This approach enables also that a naphtha side stream i.e. a naphtha bleed, be withdrawn after R2, rather than before. The latter occurs where R1 and R2 share a common fractionation unit. Withdrawing the naphtha side stream after R2 conveys a better use of the olefins and thereby a higher yield.

The operating conditions of e.g. R1 are dynamically adjusted, either manually or from automated feed-back control, suitably the latter, to maintain the process at the optimal point of operation by counteracting the continuous effect of catalyst deactivation caused by coking and dealumination, and maximizing the yield of distillate boiling range hydrocarbons, in particular hydrocarbons in the jet fuel boiling range. This dynamic adjustment of R1 upstream a first separation unit, e.g. high-pressure separator, and upstream a second separation unit, e.g. a fractionation unit, as it will also become apparent from a below embodiment, results in stable operation of these units, as the different flows therethrough are kept at a steady level, i.e. constant. Conventionally, to obtain constant product for fast deactivating processes such as catalytic deactivation, fluid bed or moving bed operation is needed. A fluid bed, for instance, is much more complicated to operate and to scale up. In contrast, a fixed bed is easier to operate, more reliable and significantly simpler to scale up, thus also more directly applicable to industrially relevant conditions and not least commercially viable.

For years, inventors across the world have been looking for ways to decrease the cost and maintain the necessary stability in jet fuel production without success. By using the oxygenate slip method of the present invention, it is possible to lessen the effects of the zeolite deactivation in the first reactor R1. That is to say that between regenerations of the zeolite, the same amount of all the product streams (C8+, C5-C7 and C4−) with the same composition will be produced. This stability of R1 is a huge advantage for the further processing of the product, such as in downstream fractionation units. Other appli-cants have used complex technologies such as fluid bed to get a similar effect; however, again, this is more complicated and makes the product more expensive. Hence, the invention not only enables producing a sizeable jet fuel proportion in one step, but also jet fuel hydrocarbons with a significant olefin content, as well as in a stable manner.

Further, as mentioned before, by the invention the energy input required for regeneration of the conversion catalyst is significantly reduced. Also completely unexpected, the properties of the coke generated in the conversion catalyst according to the present invention are different than e.g. applicant's U.S. Ser. No. 11/060,036 (WO 2019219397). In particular, the oxidation reactivity and thus burning rate of coke generated in the conversion catalyst according to the present invention is higher than e.g. applicant's U.S. Ser. No. 11/060,036 (WO 2019219397), thus enabling to start and finish at lower temperatures the regeneration of the catalyst by burning off the coke. Furthermore, not only is the content of olefins in the jet fuel range hydrocarbon fraction, such as the C8-C17, higher than in U.S. Ser. No. 11/060,036 (WO 2019219397), but also the content of olefins in the C5-C7 hydrocarbon fraction. The latter is thus made readily available, already in R1, for oligomerization into jet fuel range hydrocarbons.

Other benefits of the invention are provided in connection with one or more of below embodiments.

In an embodiment, in step d), said C5+ hydrocarbons are at least 75 wt %, such as at least 80 wt %, or at least 90 wt %, for instance 80-90 wt %, of said converted-oxygenate product. Hence, as for instance shown in appended FIG. 4, there is a high yield of C5+ hydrocarbons.

In an embodiment, in step d, at least 40 wt %, or at least 45 wt %, or least 50 wt % of said C5+ hydrocarbons are said hydrocarbons boiling in the jet fuel boiling range; suitably C8-C17 hydrocarbons. For instance, 40, 50, 60, 70, 80 or 90 wt %; such as 40-60 wt %, or 45-55 wt of said C5+ hydrocarbons are said hydrocarbons boiling in the jet fuel boiling range. Suitably, said hydrocarbons boiling in the jet fuel boiling range are C8-C19 hydrocarbons, such as C8-C17 or C8-C16 hydrocarbons. Hence, there is a high yield of C8+ hydrocarbons, e.g. C8-C17, in the C5+ hydrocarbon fraction.

The rate of converted-oxygenate product being produced is not only stable, but also shows already a product distribution comprising hydrocarbons boiling in the jet fuel boiling range, such as C8-C17 or C8-C16 hydrocarbons, for instance as olefins. Hence, there is a high selectivity towards the desired product: the hydrocarbons boiling in the jet fuel range, as for instance shown in appended FIG. 4. Again, conventionally, such hydrocarbons in the jet fuel boiling range are produced first after conducting a subsequent oligomerization of e.g. C4-C8 olefins produced during a prior oxygenate conversion. For instance, applicant's WO 2022063992 discloses in the examples oxygenate conversion (methanol to olefins, MTO) conducted with ZSM-48 in the temperature range 320-480° C., with the proportion of C8-C17 olefins produced therein being lower than 10 wt %. See also comparative example 2 in present application. Furthermore, the provision of the conversion catalyst with said P or said SAR and P content according to the present invention, further improves the process by increasing the overall olefin se-lectivity, such as by increasing the content of olefins in the C5+ fraction or in the con-verted-oxygenate product comprising C5+ hydrocarbons, i.e. olefin selectivity, as inter-mediates in the synthesis of jet fuel.

The yields represented by the above wt % (weight percentages) are as measured from the oxygenate on water-free basis, e.g. MeOH on water-free basis.

In an embodiment, at least 40 wt %, such as at least 50 wt %, or at least 60 wt %, of said hydrocarbons boiling in the jet fuel range, i.e. of the C8+ hydrocarbons, thus the C8+ product fraction, suitably of said C8-C17 hydrocarbons, are olefins. For instance, the olefin content in C8+ is 45, 50, 55, 60, 65, 70, 75, 80 wt %. Hence, as for instance shown in appended FIG. 8, not only there is a high yield of C8+ hydrocarbons, e.g. C8-C17 hydrocarbons, but also there is a high concentration of olefins. It is advantageous yet counterintuitive that there is a significant concentration of olefins in the C8+ hydrocarbons. These olefins, being already in the jet fuel range, require later hydrogenation into their saturated form. Yet, the higher the content of olefins the better since it allows further upgrading, i.e., further yield improvements. The present invention thus enables dynamically counteracting the effect of coking by temperature adjustments, allowing operation at the optimum operation point that maximize the C8+ fraction as well as its olefinic fraction.

In an embodiment, at least 4 wt %, such as at least 5 wt % or at least 6 wt %, for instance 5-10 wt %, of said hydrocarbons boiling in the jet fuel range, i.e. of the C8+ hydrocarbons, thus the C8+ product fraction, suitably of said C8-C17 hydrocarbons, are C13+ hydrocarbons. Hence, as for instance shown in appended FIG. 9, there is a shift towards the heavier C13+ hydrocarbons of the jet fuel range hydrocarbons, thus away from e.g. gasoline. The desired heavier compounds for jet fuel such as C13+ are pro-duced in a surprisingly high proportion. As shown in connection with comparative ex-ample 3 (and associated FIG. 9), the concentration of C13+ in C8+ product fraction, thus in the heavy end of jet fuel as illustrated in the typical product distributions of FIG. 7, is not only clearly visible but also highly significant compared to the prior art where values near zero wt % were measured. For instance, by the invention, the content of C13+ in said hydrocarbons boiling in the jet fuel range is at least: 6, 7, 8, 9 or 10 wt %.

Suitably, said hydrocarbons boiling in the jet fuel range are C8-C17 hydrocarbons; said C5+ hydrocarbons, i.e. the C5+ hydrocarbon fraction, comprises C5-C7 hydrocarbons; said C8-C17 hydrocarbons and said C5-C7 hydrocarbons in said C5+ hydrocarbons add up to 100 wt %.

It would be understood that in a particular embodiment, “C5+ hydrocarbons” means a hydrocarbon fraction comprising only C8-C17 hydrocarbons and C5-C7 hydrocarbons. In an embodiment, said C5+ hydrocarbons, i.e. the C5+ hydrocarbon fraction of the converted-oxygenate product, comprises C5-C7 hydrocarbons, and at least 40 wt %, such as at least 50 wt %, or at least 60 wt %, of said C5-C7 hydrocarbons, are olefins.

Hence, of the C5-C7 hydrocarbon fraction, which is not directly useful as jet fuel, in an embodiment, at least 40 wt % are olefins. These olefins are suitably later oligomerized, as explained farther above.

For the purposes of the present application, the term “first aspect of the invention” means the process of the invention. The term “second aspect of the invention” means a plant (system) i.e. process plant.

The term “comprising” includes “comprising only” i.e. “consisting of”.

The term “suitably” is used interchangeably with the term “optional” i.e. an optional em-bodiment.

The term “process/plant” means process and/or plant.

The term “and/or” means in connection with a given embodiment any of three options.

For instance, the term “to inlet of R1 and/or to inlet of R2” covers the three options: “to inlet of R”, “to inlet of R2”, “to inlet of R1 and R2”.

The term “present invention” or simply “invention” are used interchangeably with the term “present application” or simply “application”.

The term “reaction zone” means a physically delimited space where a catalytic reaction takes place and thus comprising a catalyst. For instance an adiabatic fixed bed, or a reactor comprising an adiabatic fixed bed.

The term “distillate boiling range hydrocarbons” or “distillate boiling range hydrocarbon product” means, for the purposes of the present application, C5-C30 hydrocarbons and comprises hydrocarbons boiling in the naphtha boiling range, hydrocarbons boiling in the jet fuel boiling range, hydrocarbons boiling in the diesel boiling range; optionally, a heavy hydrocarbon fraction i.e. maritime fuel.

The term “hydrocarbons boiling in the gasoline boiling range” may be used interchange-ably with the term “gasoline” and means C5-C12 hydrocarbons boiling in the range 30-210° C.

The term “hydrocarbons boiling in the naphtha boiling range may be used interchange-ably with the term “naphtha” and means C5-C9 hydrocarbons boiling in the range 30-160° C., such C5-C8 hydrocarbons, e.g. C5-C8 olefins. The term “naphtha” is sometimes used interchangeably with the term “naphtha stream”.

The term “hydrocarbons boiling in the diesel boiling range” may be used interchangeably with the term “diesel” and means C8-C25 hydrocarbons boiling in the range 120-360° C., for instance 160-360° C.

The term “hydrocarbons boiling in the jet fuel range” may be used interchangeably with the term “jet fuel hydrocarbons” or “jet fuel range hydrocarbons”, or respectively, “jet fuel” or “jet fuel range”. The term means C8-C19 hydrocarbons, such as C8-C17 or C8-C16 hydrocarbons, boiling in the range 130-300° C. The term “C8-C19” hydrocarbons is also used interchangeably with the term “C8+ hydrocarbons” or “C8+ product fraction”. The term “C13+ hydrocarbons” means C13-C19 hydrocarbons, such as C13-17 hydrocarbons. For instance, the jet fuel is sustainable aviation fuel (SAF) in compliance with ASTM D7566 and ASTM D4054. For instance, the jet fuel is in compliance with ASTM D7566.

The term boiling in a given range, shall be understood as a hydrocarbon mixture of which at least 80 wt % boils in the stated range.

The term “constant” is used interchangeably with the term “stable” and means within 10% of a given flow level, such as within 10% of a given flow rate.

Unless otherwise stated, when percentages are provided for a given stream it is meant vol. %. It would be understood that vol. % are normally used for gas streams, while wt % are normally used for liquid streams.

The term “at least a portion” of a stream means a portion of the stream or the entire stream.

Oher definitions are provided in connection with one or more of above or below embod-iments.

In an embodiment the one or more adiabatic fixed bed reaction zones R1 are provided as separate conversion reactors R1, i.e. each with its own pressure shell.

Hence, a reaction zone is a reactor; an adiabatic fixed bed reaction zone is an adia-batic fixed bed conversion reactor. For instance, in step c, one or more feed streams of one or more oxygenate compounds are introduced into the inlet of one or more downstream adiabatic fixed bed conversion reactors (R1). For instance, one or more downstream adiabatic fixed bed conversion reactors is provided as a first set (R1) of one or more adiabatic fixed bed conversion reactors operating in parallel. The parallel ar-rangement and operation enable periodic regeneration of the catalyst by burning off the coke deposited therein.

In an embodiment, in step g, said constant amount of the one or more unconverted-ox-ygenate compounds is between 1000 and 3000 ppmv, such as between 1500 and 2500 ppmv.

Within these ranges of the unconverted oxygenate compounds, such as a methanol slip at outlet (exit or effluent) R1, the highest yield of the desired jet fuel range hydrocarbons e.g. C8-C17 in C5+ hydrocarbons, are often observed. For instance, a methanol slip between 1500 and 2500 ppmv provides a normalized yield of above 98%, as shown in appended FIG. 2. Thus, the maximum jet fuel yield in C5+ hydrocarbons and the maximum C5+ yield can be found located at a conversion level corresponding to a methanol slip between 1500 to 2500 ppmv.

The term “normalized yield” means that the values are normalized by the highest yield value obtained.

The yield is provided as weight percentage (wt %) of a given hydrocarbon fraction, and is measured on water-free basis, thus as defined earlier, measured from e.g. MeOH on water-free basis.

In an embodiment, step g further comprises maintaining a constant level of conversion of the one or more oxygenate compounds of between 93 and 99.9%. Suitably, there is constant first recycle to oxygenate ratio, such as first recycle to oxygenate ratio of 5-15 w/w, for instance a first recycle-to-methanol ratio of 5-15 w/w, as recited in a below embodiment.

The process of the invention operates at a broader range of oxygenate conversion while producing a high proportion of hydrocarbons boiling in the jet fuel boiling range, compared to applicant's U.S. Ser. No. 11/060,036 (WO 2019219397) where the corresponding oxy-genate conversion range is 95-99.9%, apart from the latter being directed to gasoline production. Higher flexibility in the process of the present invention is thereby achieved.

The yields go down at lower oxygenate conversion because of loss of oxygenate, e.g. methanol, which goes unconverted through R1, thus a higher methanol (MeOH) slip.

The unconverted methanol may be reclaimed from the process condensate (water rich stream) withdrawn downstream, and routed back, e.g. via a methanol feed tank ar-ranged upstream R1.

In an embodiment, the composition of the converted-oxygenate product comprising C5+ hydrocarbons, i.e. said converted-oxygenate product, is: paraffins (P): 4-11 wt %, iso-paraffins (I): 5-30 wt %, olefins (O): 40-75 wt %, naphthenes (N): 6-15 wt %, aromat-ics (A): 4-20 wt %, in which the sum of P+I+O+N+A (PIONA) is 100 wt %.

In an embodiment, the olefin content of said C5+ hydrocarbons is at least 75 wt %, such as at least 80 wt % or at least 85 wt %.

As is well-known in the art, to determine n-paraffins, iso-paraffins, olefins, naphthenes and aromatics (i.e. PIONA composition) in a hydrocarbon mix containing hydrocarbons having boiling points in between −42 and 350° C., analytical techniques based on gas chromatography are available, e.g. in accordance with ASTM D8071—VUV-PIONA; or GCxGC-FID.

It would be understood that the art of chemistry and here in particular the art of producing hydrocarbons over zeolite-based catalysts is highly unpredictable, as the product distribution and yields is determined from a large reaction network involving hundreds of different chemical reactions, and where the choice of zeolite catalyst (pore network, pore size, acidity, etc.) and the reactor operating conditions influence the conversion and selectivity. The above PIONA distribution is, astonishingly, significantly different from the PIONA obtained when using a similar strategy to maximize yield of hydrocarbons boiling in the gasoline boiling range according to applicant's U.S. Ser. No. 11/060,036 U.S. Ser. No. 11/060,036 (WO 2019219397). Suitably also, the hydrocarbons boiling in the jet fuel boiling range are C8-C17 hydrocarbons. For instance, the content of olefins according to this citation is much lower than in the present invention, as for instance shown in ap-pended FIG. 8.

The inlet temperature in step b of the one or more downstream adiabatic fixed bed re-action zones is, in an embodiment, 275° C. or higher, and the outlet temperature in step f of the one or more downstream adiabatic fixed bed reaction zones is 475° C. or lower.

Suitably, the adiabatic temperature rise in the one or more downstream adiabatic fixed bed reaction zones is 30-100° C., thus the reaction temperature and thereby the aver-age reaction zone temperature, such as the average bed temperature, is in between the inlet and outlet temperature. The process enables flexibility in the reaction tempera-tures for the oxygenate conversion in R1. The higher the reaction temperature, the higher the content of aromatics and the higher the propensity of the conversion catalyst in R1 to deactivate due to dealumination of the zeolite of the oxygenate conversion catalyst. The lower the reaction temperature, the lower the production of aromatics not rel-evant for jet fuel, thus increasing the selectivity to the hydrocarbons relevant for jet fuel, and the lower the propensity for the oxygenate conversion catalyst to deactivate due to dealumination. More specifically, the higher the reaction temperature the lighter the hy-drocarbon products, as there is an overall change from production of C8+ hydrocarbons to production of C5+ hydrocarbons. The yield of hydrocarbons boiling in the jet fuel range is thus also lowered. Dealumination means decreasing the content of alu-mina in the zeolite, thus increasing SAR. While at first glance dealumination is un-wanted, the corresponding increase in SAR, together with the P in the conversion catalyst, turns out to provide a positive effect in terms of producing jet fuel hydrocarbons.

Too high SAR conveys however the apparent disadvantage that there is incomplete ox-ygenate conversion. The present invention provides on purpose an oxygenate conversion lower than 100%, which is beneficial, as recited above, while, in an embodiment, at the same time taking advantage of the high SAR, i.e. at least 100, such as at least 110, or above 130, together with P, for producing the desired jet fuel hydrocarbons. On the other hand, the effect of coking on the apparent methanol conversion becomes more significant at lower reaction temperature leading to an unstable process.

The temperature control according to the present invention increases the temperature to compensate for catalyst coking. Since the oxygenate slip is “constant” as shown in appended FIG. 3, upper portion, the reaction zone is maintained at same location in the catalyst bed, thus providing for stable product distribution even though the temperature is increased, as shown in FIG. 3, lower portion. Furthermore, having a high temperature at the end of the cycle does not deactivate the catalyst as fast as having a high temperature at the start of the cycle. It has namely also been found, that deposited coke actu-ally protects against steaming, as coke appears to block the acidic sites responsible for catalytic activity and thereby impeding vapour water to act with the catalyst and thereby dealuminating it.

In an embodiment, the inlet temperature in step b is 275-375° C. and the outlet temperature which is higher than said inlet temperature is 325-475° C.; for instance, the outlet temperature is 375° C. or lower, such as 370° C. or 360° C.; for instance, the inlet temperature is 275-325° C. such as 290° C. or 300° C.

While it may be desirable to operate R1 at moderate or low temperatures, such as at inlet temperature of 300° C. and outlet temperature of 375° C. or lower, there is a continuous coking of the conversion catalyst. As already described, the coking would extinguish the chemical reactions thereby leading to an unstable process. Coking is a natural and inherent phenomenon in zeolite-based catalysis which normally calls for operation at a conservatively high inlet temperature to maintain the process stable, e.g. operating the reactor with an constant high inlet temperature of e.g. 350° C. (and corre-spondingly constant exit temperature) throughout the cycle, so the methanol slip is maintained below some critical limit—depending on the downstream water purification system—for as long time as possible. The temperature level would in that case have to be chosen as a compromise between catalyst stability (high enough temperature to have reasonably long cycles before next regeneration) and product yield and composition. In the present invention, high temperature gives stability (good conversion of e.g. methanol) but low selectivity to hydrocarbons in the jet fuel range. In order to keep stable reactor operation, the inlet temperature to the reactor is adjusted, in particular gradually increased, over time to counteract the effects from that of catalyst deactivation, as shown in appended FIGS. 3 and 5. Maintaining the oxygenate slip constant exit the reactor by adjusting inlet temperature, allows operation at the optimal yield point without loss of process stability; thus, in essence, the dynamic operation strategy of the invention removes the compromise between stability and product selectivity otherwise needed for the fixed bed system. By maintaining the reaction temperature of the reactor low, catalyst deactivation as a consequence of dealumination is also significantly reduced, while at the same time enabling a stable product distribution over a catalyst cycle. While coke still is deposited, the low reaction temperature in the reactor results in deposition of coke having higher oxidation reactivity compared to coke deposited at higher reaction temperatures. This implies that regeneration of the coke deposited on the catalyst when operating according to the invention is milder i.e. burned off at lower regeneration temperatures, as already explained, hence resulting in less degree of dealumination of the catalyst during its regeneration, and thus again prolonging the catalyst lifetime.

In an embodiment, the pressure of R1, i.e. of the one or more adiabatic fixed bed reaction zones of R1, is 10-120 barg, such as 15-120 barg, e.g. 20-100 barg, or 20-80 barg. For instance, the pressure of R1 is any of: 10, 15, 20, 25, 30, 35, 40, 45, 50, 55, 60, 65, 70, 75 barg.

While changing, e.g. increasing, pressure may have an effect on downstream separation and thereby the components being recycled, also highly surprisingly, changing the pressure turns out not having any significant impact on the oxygenate conversion as well as selectivity and composition of the hydrocarbons boiling in the jet fuel range, for instance as measured by its PIONA composition. The process is thus highly pressure insensitive which is highly advantageous for the operation of the process/plant, since the flexibility is significantly increased: the selected pressure may be accommodated to at least closely match the operating pressure either upstream oxygenate production, i.e. upstream R1, such as the pressure associated with an upstream methanol synthesis which is typically conducted at high pressures e.g. 80 barg or higher, or to the operating pressure of a second set (R2) of one or more adiabatic fixed bed reaction zones, for oligomerization of in particular naphtha produced in the process. Typically, oligomerization of olefins into hydrocarbons boiling in the jet fuel range is conducted subsequently and in series with a prior oxygenate conversion for producing the olefins and at a higher pressure than the oxygenate conversion, thereby requiring the imple-mentation of associated equipment such as expensive pumps or compressors, with attendant increase in capital expenditure and energy consumption. Typically also, the ox-ygenate conversion is conducted at lower pressures to accommodate operation of the subsequent and serially arranged oligomerization at lower pressures. The prior art teaches to keep the pressure low in the reactors presumably because there is a desire to keep the aromatic slip low, whereas in the present invention the aromatic content turns out to be largely independent of pressure. It would be understood, that is generally advantageous to conduct oligomerization at high pressures, as higher yields are obtained. The present invention enables to conduct the whole operation at higher pressures than other one pressure systems found in the art.

The present invention enables therefore also high flexibility and operation of the oligomerization at higher pressures, which may be desirable for instance also for enabling a higher throughput in R1 and/or R2 with attendant reduction in equipment size, and with no need of intermediate compression of the olefins produced in the oxygenate conversion.

By the invention, said 1-D pore structure of the conversion catalyst is any of *MRE (ZSM-48), MTT (ZSM-23), TON (ZSM-22), or combinations thereof.

It would be understood that the term “*MRE (ZSM-48)” refers to zeolite type materials and means that the term “*MRE” and “ZSM-48” may be used interchangeably. The same applies for the terms MTT (ZSM-23), TON (ZSM-22).

It has been found that this type of zeolite in the catalyst, provides the best results in terms of producing the desired hydrocarbons in the jet fuel range.

A zeolite with a framework having a 10-ring pore structure means a pore circumference defined by 10 oxygens.

A 1-D pore structure means zeolites containing non-intersecting pores that are sub-stantially parallel to one of the axes of the crystal. The pores preferably extend through the zeolite crystal.

The three letter code, e.g. *MRE, for structure types are assigned and maintained by the International Zeolite Association Structure Commission in the Atlas of Zeolite Framework Types, which is at http://www.iza-structure.org/databases/or for instance also as defined in “Atlas of Zeolite Framework Types”, by Ch. Baerlocher, L. B. McCusker and D. H. Olson, Sixth Revised Edition 2007.

It would be understood that the term “ZSM-48” may be used interchangeably with the term “EU-2”.

In an embodiment, the zeolite i.e. the 1-D 10 ring pore structure zeolite, such as ZSM-48, has a silica-to-alumina ratio (SAR) of up to 240.

In an embodiment, the 1-D 10 ring pore structure zeolite is ZSM-48 with a SAR above 130, suitably at least 150, such as 150-200.

In an embodiment, the 1-D 10 ring pore structure zeolite is ZSM-48 with a SAR of at least: 110, 120, 130.

For instance, the 1-D 10 ring pore structure zeolite is ZSM-48 with a SAR of: 110, 115, 120, 125, 130, 135, 140, 145, 150, 155, 160, 165, 170, 175, 180, 185, 190, 195, or 200.

In an embodiment, R1 is operated at a weight hour space velocity (WHSV) of 0.1-5 h⁻¹, such as 0.3-4 h⁻¹, e.g. 0.5-2 h⁻¹, for instance 1, 1.5, 2 h⁻¹. It would be understood that WHSV is measured as: kg of oxygenate/kg catalyst/h.

In an embodiment, the concentration of the one or more oxygenates in step a is 10% or lower, such as 1, 2, 3, 4, 5, 6, 7, 8, 9%. For instance, the oxygenate is methanol, and the concentration is 5-10%. For instance, the pressure of R1 is 20 barg and the partial pressure of methanol (P_{MeOH}) is 1.4 barg or higher, such as 2 barg; thus a pressure in R1 of 20 barg, and P_{MeOH} of 2 barg at inlet to R1, corresponds to a methanol concentration (C_{MeOH}) at inlet to R1 of 10%.

These operating conditions may be combined together with any of the above or below embodiments. For instance, apart from the already recited pressures, temperatures and catalysts, the oxygenate is methanol, WHSV is 1 h⁻¹ and C_{MeOH} is 5-10%. The present invention, due to the possibility and flexibility in terms of operating at high pressures, enables also operation at higher partial pressures of oxygenates such as methanol.

In an embodiment, said one or more downstream adiabatic fixed bed reaction zones of said first set (R1) operate in parallel; and R1 operates as an oxygenate-to-olefins reaction zone, such as a methanol-to-olefins (MTO) reaction zone, e.g. a MTO reactor; and the process further comprises:
providing a second set (R2) of one or more adiabatic fixed bed reaction zones operating in parallel, in which R2 operates as an oligomerization reaction zone, such as an oligomerization reactor (OLI reactor);
and R1 and R2 are operated in parallel at least with respect to:
the one or more feed streams of one or more oxygenate compounds, i.e. R2 is co-fed with the one or more feed streams of one or more oxygenate compounds; and/or
a first C4− hydrocarbon fraction, as a first recycle stream, produced in downstream first separation unit; and/or
at least a portion of a naphtha intermediate product, as a second recycle stream, pro-duced in downstream second separation unit.

As already mentioned, conventionally, the oxygenates to olefins reaction zone R1 e.g. oxygenate to olefins reactor, is operated in series with a subsequent (downstream) oligomerization reaction zone R2 e.g. oligomerization reactor. The present invention further provides significant advantages in terms of simplification of the process and plant.

There is a significant reduction in capital expenditures, plant plot size, as well as operating expenditures: there is e.g. a single loop comprising for instance a single recycle compressor serving both R1 and R2, as illustrated in appended FIG. 1. The first recycle stream from the first separation unit is compressed via the single recycle compressor, thus a common or shared recycle compressor, and then suitably divided into a portion which is sent to R1 and another portion which is sent to R2.

As it also will become apparent from a below embodiment, the at least a portion of the naphtha intermediate product is suitably divided into a portion which is sent to R2 and a portion which is sent to R1; for instance, into a major portion which is sent to R2 and a minor portion which is sent to R1. The term “major portion” means more than 50% of a stream, here the stream being said at least a portion of the naphtha intermediate product, such as at least 60%, or at least 70%, or at least 80%, or at least 90% thereof. Since by the invention R1, e.g. MTO reactor, is operated in a dynamic manner that maintains both oxygenate conversion and yields stable over time, irrespectively of catalyst deactivation, the flow rates of the various fractions in the C5+ hydrocarbons being produced also become stable, as shown in appended FIG. 4. This provides also the benefit, that it is much easier to design and operate downstream units, such as downstream second separation unit, e.g. a fractionation unit, since it does not have to cope with the large variations in flow rates that otherwise would result from the change in catalyst selectivity in R1 due to e.g. coking. In addition, since the operation strategy of R1 also maintains the yield of naphtha at a stable and lowest possible level, it also en-sures a stable operation of R2, e.g. OLI reactor, with little variation in feed conditions. R2, rather than—as taught in the prior art—being merely utilized for dimerization or tri-merization of olefins produced in R1, such as dimerization of C4-C8 olefins from R1 into C8-C16 or C8-C17 olefins (jet fuel range). R2 is by the present invention suitably utilized for mainly converting naphtha being withdrawn from the second separation unit, i.e. the naphtha intermediate product, and which for instance comprises C5-C8 olefins.

In an embodiment, R1 and R2 are operated at the same pressure.

By further operating at the same pressure in R1 and R2, additional benefits are obtained by significantly reducing compression energy or the need of associated equipment such as a compressor and attendant piping, as already recited. For instance, the pressure in R1 and R2 is in the range 20-80 barg, as also recited above.

In an embodiment, the process further comprises:
h) separating in first separation unit, the converted-oxygenate product, optionally after combining with a reactor effluent from R2 to provide a combined effluent stream, into:
   said first C4− hydrocarbon fraction;
   converted oxygenate product comprising C5+ hydrocarbons of which at least 30 wt % are said hydrocarbons boiling in the jet fuel range;
   a process condensate comprising water and unconverted oxygenates;
i) recycling at least a portion of said first C4− hydrocarbon fraction, as first recycle stream, to inlet of R1 and/or to inlet of R2; suitably at a first recycle to oxygenate ratio of 5-15 w/w, such as first recycle-to-methanol ratio of 5-15 w/w.

In step h), the combining of the converted-oxygenate product from R1 with the reactor effluent from R2 enables sharing, apart from the first separation unit, the same associated cooling units prior to entering the first separation unit. The separating step h is conducted in a first separation unit, suitably a 3-phase separator or a high-pressure separator, the latter may also be referred to as high-pressure product separator. The converted-oxygenate product withdrawn from the first separation unit may be regarded as a crude distillate boiling range hydrocarbon product. The first C4-hydrocarbon fraction is suitably rich in C1-C4 paraffins and/or olefins. For instance, the first C4− hydrocarbon fraction is any of: methane, ethane, ethene (ethylene), propane, propene (propylene), butane, butene (butylene), and combinations thereof, optionally also comprising a minor portion of hydrogen, such as 1-10%.

As already recited, the first C4-hydrocarbon fraction may be recycled to R1 and/or R2. For instance, the first C4-hydrocarbon fraction, as a first recycle stream, is released at the top of a high-pressure separator, compressed by a recycle compressor and sent back to the inlet of R1 and/or R2. There are at least two benefits associated with this: first, the first recycle gas stream dilutes the reactor feed(s) and thereby controls the adiabatic temperature increase therein, which is suitably maintained in the range 30-100° C.; second, the first recycle gas stream contains unconverted light olefins such as ethene, and by recycling these components the yield of hydrocarbons boiling in the jet fuel boiling range is further increased.

The process condensate may be separated into water and unconverted oxygenates.

The unconverted oxygenates may be recycled to inlet of R1 and/or R2.

In an embodiment, the process further comprises:
j) separating in a second separation unit, e.g. said second separation unit, such as a fractionation unit, the converted-oxygenate product comprising C5+ hydrocarbons of which at least 30 wt % are said hydrocarbons boiling in the jet fuel range, into:
   an intermediate jet fuel product comprising said hydrocarbons boiling in the jet fuel boiling range, such as C8-C17 hydrocarbons;
   said hydrocarbon fraction boiling in the naphtha boiling range, such as C5-C8 hydrocarbons:
   an intermediate diesel product comprising hydrocarbons boiling in the diesel boiling range;
k) recycling the at least a portion of said intermediate naphtha product, as said second recycle stream, to inlet of R2 and/or to inlet of R1.

The second separation unit is for instance provided as one or more fractionation units.

The term “intermediate”, such as “intermediate naphtha product” or “intermediate jet fuel product” means that these streams may be further upgraded into a final product, for instance by a hydroprocessing step, such as a hydrogenation step and/or a hydrocracking step, thereby producing a final product, here respectively, a naphtha product or jet fuel product.

A portion of the distillates is thus withdrawn in the second separation unit as the intermediate naphtha product, which may for instance comprise C5-C8 hydrocarbons, suitably as C5-C8 olefins. It would be desirable to convert this naphtha stream into valuable products. The present invention provides further synergistic integration, since it has also been found that at least a portion, i.e. at least a fraction, of the intermediate naphtha product can be recycled to inlet of R1 operating according to the operating strategy discussed (and illustrated in FIG. 2-3), to increase the overall conversion of naphtha without any significant loss of catalyst stability in R1. It has also been found that at least a portion of the intermediate naphtha product, such as C5-C8 hydrocarbons therein, is converted into distillate boiling range hydrocarbons when fed into R2. For instance, by operating R2 at >20 barg, a weight hourly space velocity between 0.5 to 2 kg feed/kg cat./h, and a reaction temperature between 175-400° C., such as180-350° C. R1 works as primary reactor with jet fuel yield of e.g. >40 wt % of feed on hydrocarbon basis (exclusive water); see e.g. Table 1—Low T column, in the Examples section below. Accordingly, the intermediate naphtha product, comprising for instance said C5-C8 olefins, may further react by recycling it either back to R1 as a methanol conversion reactor, or to R2 as a dedicated olefin conversion reactor located in for instance a same synthesis loop i.e. same pressure level. R2 may thus for instance work as a “clean-up reactor” converting the reactive species of e.g. the C5-C8 hydrocarbons, in the intermediate naphtha product being recycled. R1 is suitably co-fed with naphtha to increase overall conversion, and R2 is suitably co-fed with methanol to improve catalyst stability and overall conversion, as illustrated in appended FIG. 1. Furthermore, C5-C7 hydrocarbons in the converted-oxygenate product, suitably as olefins, are also recovered as part of the intermediate naphtha product being recycled to the OLI reactor (R2).

Again, a portion of the remaining intermediate naphtha product is recycled, as a second recycle, and converted under reaction conditions tuned to favour oligomerization and facilitate production of long chained components, for instance by providing lower temperatures and lower weight hourly space velocities in R2 compared to R1, or an oligomerization catalyst which is different from the catalyst of R1. Limited amounts of oxygenates such as methanol may be introduced together with the naphtha feed in R2, since the formation of water inhibits coking and thereby provide even longer cycle lengths before regeneration is necessary. For instance, 10% or less, such as 1-10% e.g. 3, 5, 7%, of the one or more feed streams of one or more oxygenate compounds, is introduced to R2. A fraction of the naphtha may not be converted because of accumulation of low reactivity species such as paraffins outside the distillate boiling range. A fraction of the naphtha in R2 may be converted to larger hydrocarbons such as C17+ hydrocarbons suitably for e.g. diesel, and the amount will depend on the operation conditions and catalyst deactivation. To counteract the effect of catalyst deactivation of R1, the inlet temperature is adjusted/increased over time, resulting in a more stable product distribution over a catalyst cycle, as explained earlier.

In an embodiment, the process further comprises hydroprocessing e.g. in a hydroprocessing reactor, such as hydrogenating e.g. in an hydrogenation (HYDRO) reactor, and/or hydrocracking e.g. in a hydrocracking reactor, any of said: intermediate jet fuel product, intermediate naphtha product, and intermediate diesel product into, respectively, a jet fuel product, a naphtha product, and a diesel product.

Thereby, for instance by hydrogenating and/or hydrocracking, hydrocarbons boiling in the jet fuel range, such as C8-C17 hydrocarbons, of the intermediate jet fuel product, which may be predominantly present as olefins, are converted to the corresponding saturated compounds as the jet fuel product, suitably as sustainable aviation fuel (SAF) in compliance with ASTM D7566 and ASTM 1655.

The term “hydroprocessing” means any of hydrotreating, hydrocracking, hydrogenation (herein also referred to as hydrogenating), or combinations thereof. The hydroprocessing step is conducted in a hydroprocessing reactor i.e. a hydroprocessing unit, comprising a catalyst under the presence of hydrogen. For instance, hydrotreating is conducted over a hydrotreating catalyst for the removal of sulfur, oxygen, nitrogen, and metals from the hydrocarbons; hydrocracking is conducted over a hydrocracking catalyst for the cracking of hydrocarbons; hydrogenation is conducted over a hydrogenation catalyst to hydrogenate hydrocarbons. The hydrogenation conditions are well-known in the art, for instance as described in applicant's co-pending patent application EP 22152691.6. Hydrotreating and hydrocracking are also well-known in the art. For instance, applicant's WO 2021180805 discloses associated catalysts and operating conditions.

In an embodiment, step j further comprises:
separating a second C4− hydrocarbon fraction as an off-gas stream, and combining at least a portion of the off-gas stream with said first C4− hydrocarbon fraction to form part of said first recycle stream.

The off-gas stream is removed at the top of the fractionation unit(s) downstream R1, R2, e.g. a distillation column, and may be introduced back into R1 and/or R2 together with the first C4− hydrocarbon fraction recycled from top of the upstream separator. The benefits of recycling such light gasses (C4− hydrocarbon fraction) have been discussed earlier.

In another general embodiment according to the first aspect (process) of the invention, there is also provided a process for the conversion of oxygenates to hydrocarbons boiling in the jet fuel boiling range, comprising the steps of continuously:
a) providing one or more feed streams of one or more oxygenate compounds;
b) heating the one or more feed streams to an inlet temperature of a first set (R1) of one or more downstream adiabatic fixed bed reaction zones;
c) introducing the one or more heated feed streams into inlet of R1;
d) converting in the one or more adiabatic fixed bed reaction zones of R1 the one or more heated feed streams in the presence of a conversion catalyst comprising a zeolite with a framework having a 10-ring pore structure, said 10-ring pore structure being a unidimensional (1-D) pore structure, to a converted-oxygenate product of which at least 30 wt % are said hydrocarbons boiling in the jet fuel boiling range; wherein said 1-D pore structure is any of *MRE (ZSM-48), MTT (ZSM-23), TON (ZSM-22), or combinations thereof, and the content of phosphorous (P) in the conversion catalyst is 0.1-3 wt % (0.1-3 wt % P);
e) withdrawing from the one or more adiabatic fixed bed reaction zones the converted-oxygenate product;
f) determining at outlet of the one or more adiabatic fixed bed reaction zones an amount of one or more unconverted oxygenate compounds in the withdrawn con-verted-oxygenate product;
g) continuously adjusting the inlet temperature of the one or more feed streams in step b to maintain a constant amount of the one or more unconverted-oxygenate com-pounds as determined in step f of between 10 and 4000 ppmv.

The surprising high proportion of jet fuel hydrocarbons is thus here defined in terms of the converted-oxygenate product exiting R1, instead of in terms of the C5+ hydrocarbons in the converted-oxygenate product exiting R1.

It would thus be understood that in step d), the converted-oxygenate product may be recited as:
I) a converted-oxygenate product comprising C5+ hydrocarbons of which at least 30 wt % are said hydrocarbons boiling in the jet fuel boiling range;
or
II) a converted-oxygenate product of which at least 30 wt % are said hydrocarbons boiling in the jet fuel boiling range.

In an embodiment according to the former (I), as already recited, at least 35 wt %, or at least 40 wt %, or at least 45 wt %, or at least 50 wt % of said C5+ hydrocarbons in the converted-oxygenate product are said hydrocarbons boiling in the jet fuel boiling range; i.e. C8+ hydrocarbons (C8+ product fraction), suitably C8-C17 hydrocarbons.

In an embodiment according to the later (II), i.e. according to the another general em-bodiment of the invention of the first aspect of the invention, at least 35 wt %, or at least 40 wt %, or at least 45 wt %, or at least 50 wt % of said converted-oxygenate product, are said hydrocarbons boiling in the jet fuel boiling range i.e. C8+ hydrocarbons (C8+ product fraction), suitably C8-C17 hydrocarbons.

In an embodiment, at least 40 wt %, such as at least 50 wt %, or at least 60 wt %, of said hydrocarbons boiling in the jet fuel range, i.e. of the C8+ hydrocarbons (C8+ product fraction), suitably of said C8-C17 hydrocarbons, are olefins. For instance, the olefin content in C8+ is 45, 50, 55, 60, 65, 70, 75, 80 wt %. It is understood, that since the olefins are defined in terms of the hydrocarbons boiling in the jet fuel range, the recited weight percentages (wt %) are irrespective of the converted-oxygenate product being defined according to the above-mentioned recital I or II.

Any of the embodiments and associated benefits of the first general embodiment of the first aspect of the invention (process) may be used in connection with the another general embodiment of the first aspect of the invention, and vice versa.

In a second aspect, the present invention encompasses also a plant (system).

Hence, in a first general embodiment of the second aspect of the invention, there is provided a plant for converting oxygenates to hydrocarbons boiling in the jet fuel boiling range, the plant (10) comprising:
a first set (R1) of one or more adiabatic fixed bed reaction zones arranged in parallel and configured to operate as an oxygenate-to-olefins reaction zone, such as a MTO re-action zone, e.g. a MTO reactor; said one or more adiabatic fixed bed reaction zones comprising a conversion catalyst comprising a zeolite with a framework having a 10-ring pore structure, said 10-ring pore structure being a unidimensional (1-D) pore structure; wherein said 1-D pore structure is any of *MRE (ZSM-48), MTT (ZSM-23), TON (ZSM-22), or combinations thereof, and the content of phosphorous (P) in the conversion catalyst is 0.1-3 wt % (0.1-3 wt % P);
said one or more adiabatic fixed bed reaction zones arranged to at least receive: one or more feed streams (1, 1′) of one or more oxygenate compounds and provide: a con-verted-oxygenate product (5′) comprising C5+ hydrocarbons of which at least 30 wt % are said hydrocarbons boiling in the jet fuel boiling range; or a converted-oxygenate product (5′) of which at least 30 wt % are said hydrocarbons boiling in the jet fuel boiling range;
optionally:
   a second set (R2) of one or more adiabatic fixed bed reaction zones arranged in parallel and configured to operate as an oligomerization reaction zone, such as an oligomerization reactor (OLI reactor); said one or more adiabatic fixed bed reaction zones arranged to at least receive: at least a portion (13′) of a naphtha intermediate product (13) produced in downstream second separation unit (14); and provide a reactor effluent (5″);
   said R1 and R2 being arranged in parallel at least with respect to:
      the one or more feed streams (1, 1′) of one or more oxygenate compounds; and/or
      a first C4− hydrocarbon fraction (3), as a first recycle stream, produced in downstream first separation unit (12); and/or
      at least a portion (13′) of naphtha intermediate product (13), as a second recycle stream, produced in downstream second separation unit (14);
   a first separation unit (12) arranged to at least receive: the converted-oxygenate product (5′), suitably a combined effluent stream (5′″) which combines said converted-oxy-genate product (5′) and said reactor effluent (5″); and provide: a process condensate (9) comprising water and unconverted oxygenates, converted-oxygenate product (7), said first C4− hydrocarbon fraction (3), as said first recycle stream;
   a compressor (4) arranged to recycle at least a portion (3′, 3″) of said first C4− hydrocarbon fraction (3), as first recycle stream, to inlet of R1 and/or to inlet of R2.

In another general embodiment of the second aspect, the present invention encompasses also a plant (system) for carrying out the process of any of the above embodiments.

Hence, there is provided a plant for carrying out the process according to any of the above process embodiments according to the first aspect of the invention, the plant (10) comprising:
a first set (R1) of one or more adiabatic fixed bed reaction zones arranged in parallel and configured to operate as an oxygenate-to-olefins reaction zone, such as a MTO reaction zone, e.g. a MTO reactor; said one or more adiabatic fixed bed reaction zones comprising a conversion catalyst comprising a zeolite with a framework having a 10-ring pore structure, said 10-ring pore structure being a unidimensional (1-D) pore structure; wherein said 1-D pore structure is any of *MRE (ZSM-48), MTT (ZSM-23), TON (ZSM-22), or combinations thereof, and the content of phosphorous (P) in the conversion catalyst is 0.1-3 wt % (0.1-3 wt % P);
said one or more adiabatic fixed bed reaction zones arranged to at least receive: one or more feed streams (1, 1′) of one or more oxygenate compounds and provide: a con-verted-oxygenate product (5′) comprising C5+ hydrocarbons of which at least 30 wt % are said hydrocarbons boiling in the jet fuel boiling range; or a converted-oxygenate product (5′) of which at least 30 wt % are said hydrocarbons boiling in the jet fuel boiling range;
optionally:
   a second set (R2) of one or more adiabatic fixed bed reaction zones arranged in parallel and configured to operate as an oligomerization reaction zone, such as an oligomerization reactor (OLI reactor); said one or more adiabatic fixed bed reaction zones arranged to at least receive: at least a portion (13′) of a naphtha intermediate product (13) produced in downstream second separation unit (14); and provide a reactor effluent (5″);
   said R1 and R2 being arranged in parallel at least with respect to:
      the one or more feed streams (1, 1′) of one or more oxygenate compounds; and/or
      a first C4− hydrocarbon fraction (3), as a first recycle stream, produced in downstream first separation unit (12); and/or
      at least a portion (13′) of naphtha intermediate product (13), as a second recycle stream, produced in downstream second separation unit (14);
   a first separation unit (12) arranged to at least receive: the converted-oxygenate product (5′), suitably a combined effluent stream (5′″) which combines said converted-oxy-genate product (5′) and said reactor effluent (5″); and provide: a process condensate (9) comprising water and unconverted oxygenates, converted-oxygenate product (7), said first C4− hydrocarbon fraction (3), as said first recycle stream;
   a compressor (4) arranged to recycle at least a portion (3′, 3″) of said first C4− hydrocarbon fraction (3), as first recycle stream, to inlet of R1 and/or to inlet of R2.

Any of the embodiments and associated benefits of the first general embodiment of the second aspect of the invention (plant) may be used in connection with the another general embodiment of the second aspect of the invention, and vice versa.

Any of the embodiments and associated benefits in connection with the first aspect of the invention (process) and associated benefits may be used in connection with the second aspect of the invention (plant), and vice versa.

### BRIEF DESCRIPTION OF THE FIGURES

FIG. 1 shows a simplified block type diagram of the process and plant according to an embodiment of the invention and various process integrations.

FIG. 2 shows the normalized yields (%) of C5+ hydrocarbons and jet fuel hydrocarbons (C5-C17) of R1 at any given methanol slip of R1. Solid curve: trend line for C5+ hydrocarbons. Stippled line: trend line for C8-C17 hydrocarbons.

FIG. 3 shows an automated inlet temperature strategy to maintain methanol slip constant of R1. R1 is an adiabatic fixed bed reactor, as illustrated in FIG. 1.

FIG. 4 shows the R1 yields of liquid product (C5+) and distillate boiling range hydrocarbon product as fraction of the liquid product (C8-17 in C5+). R1 is an adiabatic fixed bed reactor operated in once-through mode in a pilot scale plant.

FIG. 5 shows the loss of catalyst activity due to dealumination for R1 loaded with ZSM-48. R1 is an adiabatic fixed bed reactor. The activity decay has been calculated from akinetic model taking the effect of temperature into account.

FIG. 6 shows a parity plot of deactivation model used in FIG. 5, and actual deactivation data from loss of activity of the zeolite during pilot plant operation, where the reactor operates under commercially relevant conditions.

FIG. 7 shows a typical carbon number distribution of gasoline, jet fuel and diesel.

FIG. 8 shows a comparative example of olefin content in the C8+ product fraction (jet fuel hydrocarbons) from the oxygenate conversion reactor.

FIG. 9 shows a comparative example of the concentration of the heavier molecules C13+ in the C8+ product fraction (jet fuel hydrocarbons) from the oxygenate conversion reactor.

### DETAILED DESCRIPTION

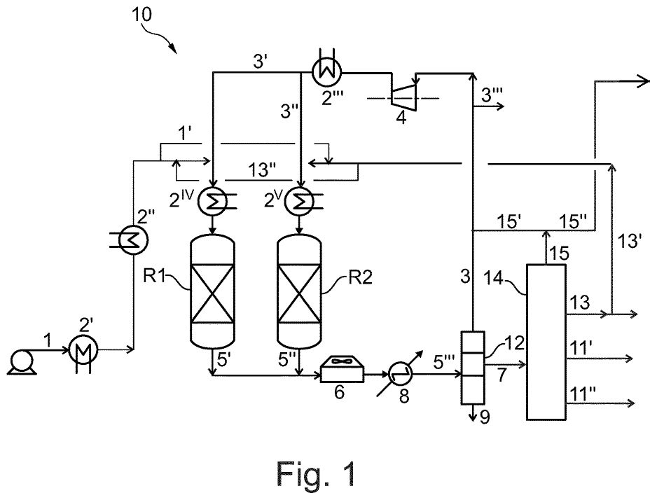

With reference to FIG. 1, a schematic layout of an embodiment of the process/plant 10 is shown. A feed of liquid oxygenate(s) such as methanol 1 is pumped up from a methanol feed tank (not shown) to operating pressure of e.g. at least 20 barg, pre-heated in heat exchangers 2′, 2″, mixed with a portion 3′ of first recycle stream 3′, further pre-heated in heat exchanger 2″ and then introduced into the first oxygenate conversion reactor set R1, here illustrated as one adiabatic fixed bed reactor comprising one fixed catalyst bed, and which is tuned to facilitate methanol conversion and formation of distillate boiling range hydrocarbons. The adiabatic fixed bed reactor R1 is operated such that the reaction temperature is maintained as low as possible for as long time as possible. This is achieved by combining frequent measurements of the methanol effluent 5′ at R1-outlet (e.g. by GC) and a programmable feed-back action adjusting the inlet temperature to the reactor. In that way, the effect of catalyst coking is continuously counteracted by automated temperature adjustments. Since the catalyst of the reactor set R1 is re-generated regularly, for instance once a month, several reactors operating in parallel are necessary during continuous production, whereby the reactor shifts between the reaction mode and regeneration mode. The converted-oxygenate product is withdrawn as reactor effluent 5′, combined with reactor effluent 5″ from R2 suitably arranged in parallel, as explained below. The combined effluent is cooled down in air cooler 6 and heat exchanger 8 using e.g. cooling water. Water and unconverted methanol is withdrawn in process condensate stream 9 from first separation unit 12, such as a high-pressure separator 12. The process/plant may thus further comprise a second set (R2) of one or more adiabatic fixed bed reaction zones operating in parallel, in which R2 operates as an oligomerization reaction zone, such as an oligomerization reactor (OLI reactor). Further, R1 and R2 are suitably operated in parallel with respect to: the one or more feed streams 1, 1″ of one or more oxygenate compounds; and/or in parallel with respect to a first C4− hydrocarbon fraction 3, as a first recycle stream, being produced in the downstream first separation unit 12; and/or in parallel with respect to an intermediate naphtha product 13′, 13″, as a second recycle stream, being produced in a downstream second separation unit 14, e.g. a fractionation unit.

The first C4-hydrocarbon fraction 3 (first recycle stream) is released at the top of the first separation unit 12, compressed by recycle compressor 4 and sent back to the inlet of R1, R2 as recycled gas 3, 3′, 3″. A purge, off-gas stream 3′″, may also be derived there-from. The recycle gas stream dilutes the R1, R2 feed and thereby controls the adiabatic temperature increase therein. In addition, the recycle gas stream contains unconverted light olefins. By recycling these components, the yield of distillate boiling range hydrocarbon product is further increased. From the first separation unit 12, a converted-oxy-genate product comprising C5+ hydrocarbons of which at least 30 wt %, such as at least 40 wt %, at least 45 wt % or at least 50 wt % are said hydrocarbons boiling in the jet fuel range, is withdrawn as a crude distillate boiling range hydrocarbon product 7. From the first separation unit 12, a bottom stream is withdrawn as the process condensate 9 comprising water and unconverted oxygenates. The crude distillate 7 is sent to the second separation unit 14, here simply illustrated as a fractionation unit, such as a distillation column, where the crude distillate 7 is separated into: intermediate jet fuel product 11′, intermediate naphtha product 13, and a second C4− hydrocarbon fraction as an off-gas stream 15. The intermediate jet fuel product 11′ can be hydrogenated directly in a downstream unit (not shown) to produce a jet fuel product. Suitably also, an intermediate diesel product 11″ may be withdrawn from the distillation column 14 as well. The off-gas stream 15 is removed at the top of distillation column 14 and a portion thereof may be introduced back into the loop (oxygenate conversion loop) as stream 15′, while the other portion 15″ is removed from the process/plant, as further illustrated in FIG. 1.

In order to convert the reactive species in the naphtha range fraction for increasing the yield of the distillate boiling range fraction, the naphtha fraction, suitably as a liquid, is recycled. Thus, more specifically, a portion 13′, 13″ of the intermediate naphtha product 13, which for instance comprises C5-C8 hydrocarbons, suitably C5-C8 olefins, is recycled to R1 and/or R2. To ensure full conversion of the reactive part of the naphtha fraction, the naphtha being recycled may be divided between R1 and R2 suitably operating at different conditions. At least a portion of the naphtha can be recycled as stream 13″ and co-fed together with the methanol feed 1 to increase the yield of long chained molecules in R1. At least a portion of the remaining naphtha can be recycled to R2 as stream 13′ and converted under reaction conditions tuned to favour oligomerization of the e.g.

C5-C8 hydrocarbons, and facilitate production of long chained components, for instance by providing a lower temperature and lower weight hourly space velocity in R2. Hence, R1 is suitably a methanol to olefins (MTO) reactor and R2 is an oligomerization (OLI) reactor. Hence, as described above, R1 and R2 are suitably arranged in parallel and operate in parallel. Limited amounts of methanol feed 1′ may be introduced together with the recycled naphtha of stream 13′ in R2.

### EXAMPLES

The conversion from oxygenates into distillate boiling range hydrocarbons is highly surprisingly achieved in a single oxygenate conversion step utilizing reaction synergies dis-covered from experimental tests in a 0.6 bpd (barrels per day) pilot plant. The findings were made during pilot tests where the zeolite catalyst ZSM-48 was tested under industrially realistic reactor operating conditions. The reactor diameter-to-catalyst particle ratio in the experimental setup was about 40, which is more than sufficient to avoid undesired wall effects. In the pilot, the feed flow rate to the reactor was set to match the industrial relevant mass fluxes, thereby ensuring the correct mass and energy transfer between the bulk fluid phase and the surface of the catalyst particles. Application of commercial catalyst particle dimensions combined with industrial Reynolds numbers thus provide an overall transport limitation, from the bulk of the fluid to the active catalytic sites, like what is obtained for the commercial reactors. The pilot simulated the entire oxygenate conversion loop including recycle of light gasses to the inlet of the reactor. Various pressure levels, space velocities, recycle-to-make up gas ratios, and temperature levels were tested. The adiabatic catalyst bed temperature was measured along the entire bed length with a 4 mm diameter multipoint Rosemount K-type thermoelement (273.15 K up to 1373.15 K) in direct contact with the catalyst bed material. Inlet, exit, and recycle stream rates are measured by Coriolis mass flow meters. These together with composition analysis, form full mass balance closure by error smoothing of the entire oxygenate conversion loop.

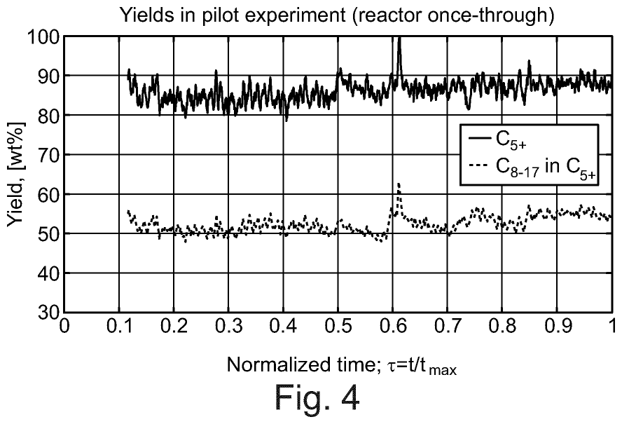

From the experimental pilot results, it was i.a. found, that when operating on methanol feed at high pressure (e.g. at least 20 barg) and low reaction temperature such as outlet temperature below 370° C., a large fraction, i.e. about 50 wt % or more, of the C5+ hydrocarbons being produced, was astonishingly already in the jet fuel boiling range. This high yield of larger hydrocarbon species already in product range was completely unexpected with this catalyst from numerous laboratory scale experiments as well as from the open literature. The total liquid yield (C5+) at these conditions was about 85 wt % on hydrocarbon basis excluding water, i.e. on water-free basis, see FIG. 4. The gas phase product (C4−) was mainly butane and propane and some light olefins. If the temperature was increased the result was an observable sharp decrease in both liquid yield and the fraction of the distillate boiling range hydrocarbons which include said hydrocarbons boiling in the jet fuel range. Measured yields at both temperatures (low and high temperature) are provided in below Table 1. On the other hand, if the temperature remained at the low level, such as 290° C., the continuous coking of the catalyst would extinguish the chemical reactions thereby leading to an unstable process. Consequently, to keep stable reactor operation, the inlet temperature to the catalyst bed was adjusted/increased over time to counteract the effects of that mechanism of catalyst deactivation. Coking is counteracted by continuous and appropriate increase of inlet temperature, yet if the temperature becomes too high, catalyst deactivation by other mechanism, namely dealumination of the zeolite, takes place at an accelerated rate. The reactor inlet temperature needed to balance the methanol slip exiting the reactor at the same constant value resulted in a fairly constant liquid product yield (C5+ hydrocarbons) as well the yield of the distillate boiling range hydrocarbons, as also shown in FIG. 4.

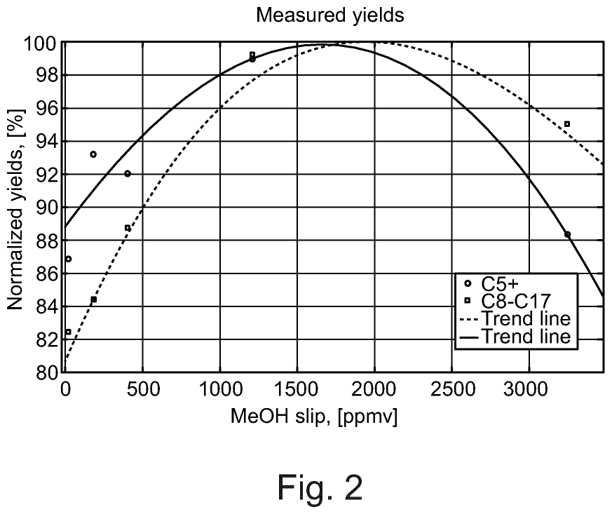

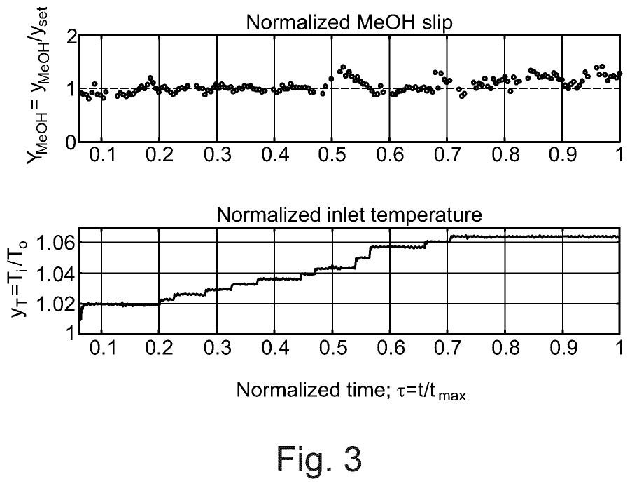

The normalized yields plotted vs the methanol slip are shown in FIG. 2. The term “normalized yield” means that the values are normalized by the highest yield value obtained (highest yield is for instance here about 55 wt %). The yield of C5+ hydrocarbons of which for instance about 45 wt % are jet fuel hydrocarbons (in FIG. 2 represented as C8-C17)—see also FIG. 4 and below Table 1—, is a function of the methanol conversion and selectivity to said C5+ hydrocarbons. The temperature increase and corresponding methanol slip are shown in FIG. 3 and the obtained product yields are provided in FIG. 4.

Operating with a constant methanol slip of between 10 and 4000 ppmv, as shown in FIG. 2, thus using the temperature strategy shown in FIG. 3, allows a stable high production of final range distillate product already in the methanol conversion reactor R1. FIG. 239 shows, along with FIGS. 3 and 4, the advantage in terms of yields of e.g. C8-C17 hydrocarbons, by not operating with complete conversion of methanol (complete conversion means methanol slip=0 ppmv). By maintaining a given methanol slip in the range 10-4000 pppmv, the optimum yield for C8-C17 is captured.

TABLE 1
Measured yields from methanol on hydrocarbon basis exclusive water
from pilot plant operation. Low temperature operation in the methanol
conversion reactor favours production of distillate boiling range
hydrocarbons. These yields are valid for “once-though”
operation, i.e. no recycle of heavier hydrocarbon species.

| | |
|---|---|
| Low T operation | High T operation |

| | |
|---|---|
| Hydrocarbon fraction | Yields, [wt %] |

| | | |
|---|---|---|
| C4− | 15 | 25 |
| C5+ | 85 | 75 |
| C8-C17 | 45 | 30 |
| C5-C7 | 40 | 45 |

Olefin content in C8-C17 hydrocarbon fraction, Low T operation: 62 wt %.

In connection with Table 1, in the pilot experiments during the “Low T operation”, the inlet temperature was increased as per invention, and at a point in time where the inlet temperature was about 310° C., a step change was made in the temperature up to 350° C. (“High T operation”). A decrease in yield was observed, as shown in the table.

It is observed from the Low T operation column (invention) that, for instance, the C5+ hydrocarbons, i.e. the C5+ hydrocarbon fraction, is 85 wt % of said converted-oxygenate product, while the remaining 15 wt % is C4− hydrocarbons, i.e. the C4− hydrocarbon fraction. It is also observed that the C5+ hydrocarbons, for instance here 85 wt % of the converted-oxygenate product, contains 45 wt % hydrocarbons in the jet fuel range, here specifically 45 wt % C8-C17 hydrocarbons. This represents more than 50 wt % of the C5+ hydrocarbons. (45/85×100=53 wt %). The remaining C5-C7 hydrocarbons, i.e. the C5-C7 hydrocarbon fraction, of the C5+ hydrocarbons, represents thereby less than 50 wt %, more specifically 47 wt %. In contrast, when allowing the operation to complete conversion of methanol, the Hight T operation column not only shows lower yield of C5+ hydrocarbons, but also the proportion of C5-C7 in the C5+ hydrocarbons is higher than the proportion of the more desirable C8-C17 hydrocarbons.

Alternatively, it is also observed from Table 1, that the jet fuel range hydrocarbons from R1 is e.g. 45 wt % of the converted-oxygenate product; here specifically 45 wt % C8-C17 hydrocarbons (Low T operation column of Table 1). In additional pilot tests, the produced liquid olefin rich product was after separation pumped back to the inlet reactor. Two tests were made. In one test the olefin rich product was introduced into the synthesis loop together with methanol feed, and in another test the olefin rich product was introduced alone. From the experiments it was found that a fraction corresponding to at least all the hydrocarbons in the naphtha range can be recycled to inlet of the methanol conversion reactor operating according to the strategy discussed and illustrated in FIG. 2, 3, to increase the overall conversion of naphtha without any significant loss of catalyst stability.

It was further found, that at least a fraction of the naphtha range hydrocarbons can be converted into distillate boiling range hydrocarbons when fed into an adiabatic reactor (corresponding to R2) operating at high pressure, e.g. at least 20 barg, a weight hourly space velocity between 0.5 to 2 kg feed/kg cat./h, and a reaction temperature between 180-350° C. It would be understood that the reaction temperature lies within the range of the inlet and outlet temperature of the adiabatic fixed bed reactor, for which the adiabatic temperature rise is e.g. 30-100° C.

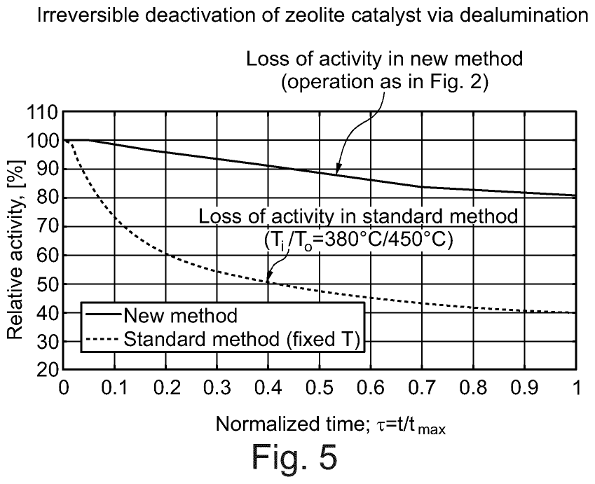

FIG. 5 shows a plot of irreversible deactivation of the zeolite catalyst via dealumination.

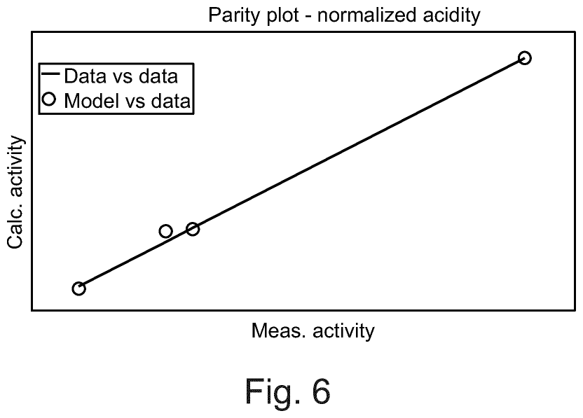

The plot shows relative activity of the catalyst as a function of the normalized time. The upper solid line shows the relative activity (loss of activity) according to the new method, i.e. present invention, thus with active regulation of methanol slip as per FIG. 2, 3, whereas the lower stippled line shows the loss of activity according to the standard method, i.e. whereby the temperature is fixed, for instance temperature at inlet of 380° C., temperature at outlet 450° C., without active regulation of methanol slip. The activity decay shown in FIG. 5 has been calculated from a kinetic model taking the effect of temperature into account. FIG. 6 (X-axis: measured activity; Y-axis: calculated activity) shows that the kinetic model—kinetic evaluation of the loss of Brönsted acidity (catalyst activity)—, in the figure denoted as “model” is reliable.

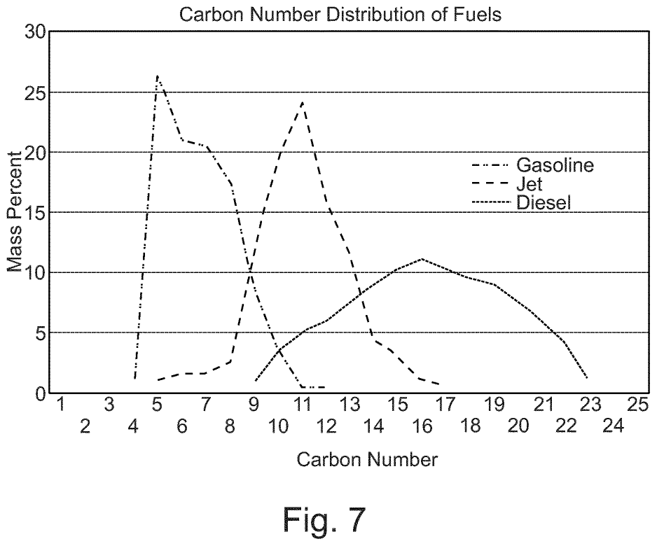

FIG. 7 shows a typical carbon number distribution of gasoline (left hand curve in the figure), jet fuel (jet, center curve in the figure) and diesel (right hand curve in the figure), according to the prior art. Another plot of carbon number distributions may be retrieved from:
https://www.researchgate.net/figure/Carbon-number-distribution-of-petroleum-fuels_fig1_267420915

This figure serves to show that the product pool for gasoline is very different from that of jet fuel. The production of jet fuel relates to a completely different field: different catalyst, different chemistry and kinetics, as well as a different product.

Effect of P in the conversion catalyst (oxygenate conversion catalyst): The below table (Table 2) shows results of MTO tests run in a fixed catalyst bed (fixed bed) reactor at 20 bar, 280-480° C. isothermally and WHSV=2 h⁻¹. Table 2 shows the MTO product distribution simply in terms of the olefin content of C5+ hydrocarbons produced, when providing:
a) a relatively low SAR in a conversion catalyst being 50-60 wt % extruded with alumina; the conversion catalyst (catalyst) comprises ZSM-48 with SAR of 102 and with no P in the catalyst;
b) a higher SAR in a catalyst being 50-60 wt % extruded with alumina; the catalyst comprises ZSM-48 with SAR of 152 and with no P in the catalyst;
c) a higher SAR in a catalyst being 50-60 wt % extruded with alumina; the catalyst comprises ZSM-48 with SAR of 152 and with 1 wt % P in the catalyst after P-impregnation of the extrudates.

The SAR is based on Si and Al X-ray fluorescence (XRF) analysis, as is well-known in the art.

TABLE 2
Content of olefins in C5+ hydrocarbons

| | Temperature | Olefins in C5+ |
|---|---|---|
| Test run | (° C.) | hydrocarbons (wt %) |
| a) | 360 | 67.5 |
| b) | 360 | 76.1 |
| c) | 360 | 80.2 |

By (c), the olefin content and thereby olefin selectivity of the product is at least 80 wt % of the C5+ hydrocarbons (C5+ fraction), thus significantly higher than any of the tests where no P is provided in the catalyst.

### EXAMPLES—COMPARATIVE

### Comparative Example 1

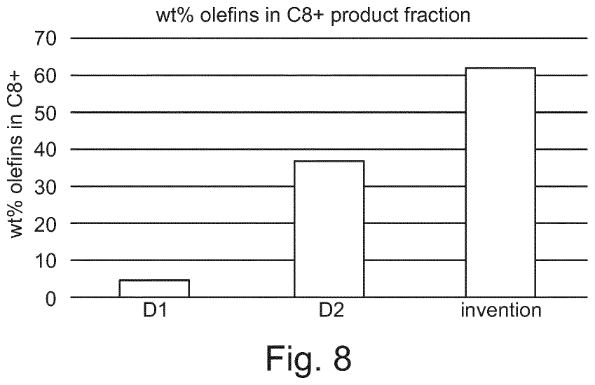

A comparative example was conducted to show the highly unexpected results of the present invention with respect to the prior art. Hence, FIG. 8 shows the olefin content in the C8+ product fraction (more specifically the C8-C17 hydrocarbon fraction) from the oxygenate conversion reactor according to the present invention with respect to the afore mentioned prior art (D1: applicant's US20210002557 corresponding to WO 2019219397; D2: applicant's WO 2022063994 with oxygenate conversion using the same ZSM-48 zeolite).

FIG. 8 shows that there is a surprising cumulative effect in the present invention that is not inferred from D1 and D2, alone or in combination. By the present invention, not only it is now possible to obtain a high C8+ yield, e.g. C8-17 yield, but also with high olefin content. At least 30 wt % of said C5+ hydrocarbons, such as at least 50 wt % of said C5+ hydrocarbons, as measured from MeOH on water-free basis; or alternatively, the at least 30 wt % hydrocarbons boiling in the jet fuel range of the converted-oxygenate product; or more generally, the hydrocarbons boiling in the jet fuel range (C8+ product fraction), also have a high concentration of olefins: at least 40 wt % olefins in said C8+ product fraction, such as above 60 wt % or above 62 wt % olefins as shown in the right-hand column of the figure. It is understood that the C8+ product fraction represents the jet fuel range hydrocarbons. This C8+ olefin content (wt % olefins in C8+) is next to nothing in D1, namely <5 wt %). D2 does not use temperature adjustments to control the methanol conversion as in the present invention, and the wt % content of olefins in C8+ is significantly lower.

### Comparative Example 2

As recited earlier, the product distribution of the present invention is also completely different to applicant's WO 2022063992. This citation discloses in the examples oxy-genate conversion (methanol to olefins, MTO) conducted with the same ZSM-48 in the temperature range 320-480° C., with the proportion of C8-C17 olefins produced therein being lower than 10 wt %. A subsequent oligomerization of e.g. C4-C8 olefins produced from the oxygenate conversion is thus required to increase the yield of product into the desired olefins in the jet fuel range.

In contrast thereto, the rate of converted-oxygenate product being produced by the present invention is not only stable, but also shows already a product distribution comprising a higher proportion of hydrocarbons boiling in the jet fuel boiling range. There is ahigh selectivity towards these hydrocarbons, again as shown in appended FIG. 4. Further, as shown in above Table 1—Low T operation, the corresponding content of C8− C17 olefins of the present invention is 28 wt % (45×62%=28 wt %—see also FIG. 8).

Hence, almost a factor of 3 higher than according to WO 2022063992.

### Comparative Example 3

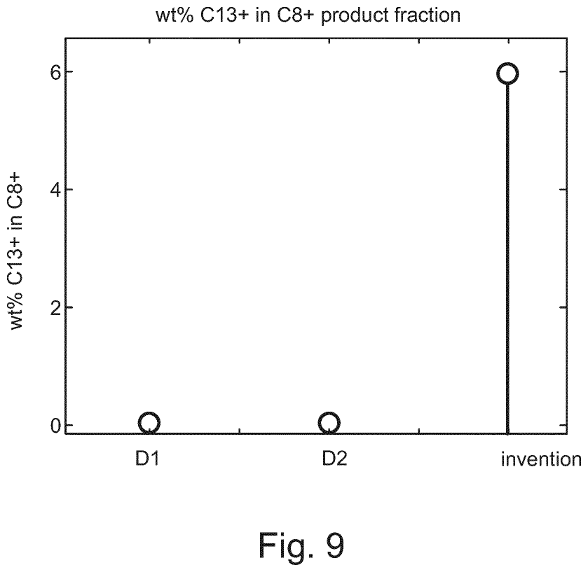

FIG. 9 shows a comparative example of the concentration of the desired heavier molecules C13+, hence in the heavy end of jet fuel, according to the present invention and the prior art (D1: applicant's WO 2019219397; D2: applicant's WO 2022063994). This figure clearly shows that the concentration of these molecules which are in the heavy end of jet fuel—see for instance FIG. 7—is next to nothing in D1, where a content of about 0.02 wt % C13+ in the C8+ product fraction is measured, while in D2 a content of 0 wt % was measured (below detection limit) even for C10+. In contrast thereto, the present invention shows here at least 6 wt % C13+ in the C8+ product fraction. The C13+ are hydrocarbons boiling in the jet fuel range, i.e. C13-C19, such as C13-C17.

The invention provides at least the following benefits: Constant, i.e. stable, product composition even though fixed bed reactors are applied.

Conventionally, to obtain constant product for fast deactivating processes fluid bed operation is needed. At the same time, a sizeable jet fuel proportion is produced in one step in R1.

The invention enables dynamically counteracting the effect of coking by temperature adjustments, allowing operation at the optimum operation point that maximize the C8+ product fraction, e.g. C8-C7, and its olefinic fraction. The higher the content of olefins the better since it allows further upgrading, i.e. further yield improvements.

Significant prolonged catalyst lifetime. The mild temperature operation in R1 reduces significantly the catalyst deactivation rate due to coking and dealumination.

Further to the latter, the mild or low temperature operation results also in deposition of coke with higher oxidation reactivity compared to coke deposited at higher reaction temperature. This implies, that regeneration of the coke deposited on the catalyst when operating according to the invention is more easily burned off at lower regeneration temperatures, resulting in less degree of dealumination of the zeolite during regeneration, and again prolonging the catalyst lifetime. A kinetic evaluation of the loss of Brönsted acidity (catalyst activity) for operating ZSM-48 according to this new process compared to the conventional MTO operation at constant and high temperatures is provided in FIG. 5-6. The loss of activity of the catalyst is significantly lower when operating according to the new method of the invention, as shown in FIG. 2, 3, compared to a conventional (standard) method in which a fixed temperature is applied.

Furthermore, the provision of the conversion catalyst with said SAR and P content, further improves the process by increasing the overall olefin selectivity, such as by increasing the content of olefins in the C5+ fraction, i.e. olefin selectivity, as intermediates in the synthesis of jet fuel.

Another benefit of the present invention is the increased commercial viability of the pro-cess/plant. The prior art teaches operation of fixed bed reactors at different pressures requiring more expenditure, or at lower pressures giving reduced effectivity particularly in the oligomerization (OLI reactor). By providing a fixed bed reactor—based pro-cess/plant that is pressure independent in the methanol conversion (MTO reactor), a much superior solution than process/plants according to the prior art is achieved.

1. Process for the conversion of oxygenates to hydrocarbons boiling in the jet fuel boiling range, comprising the steps of continuously:
a) providing one or more feed streams of one or more oxygenate compounds;
b) heating the one or more feed streams to an inlet temperature of a first set (R1) of one or more downstream adiabatic fixed bed reaction zones;
c) introducing the one or more heated feed streams-) into an inlet of R1;
d) converting in the one or more adiabatic fixed bed reaction zones of R1 the one or more heated feed streams in the presence of a conversion catalyst comprising a zeolite with a framework having a 10-ring pore structure, said 10-ring pore structure being a unidimensional (1-D) pore structure, to a converted-oxygenate product comprising C5+ hydrocarbons of which at least 30 wt % are said hydrocarbons boiling in the jet fuel boiling range; wherein said 1-D pore structure is any of *MRE (ZSM-48), MTT (ZSM-23), TON (ZSM-22), or combinations thereof, and the content of phosphorous (P) in the conversion catalyst is 0.1-3 wt % (0.1-3 wt % P);
e) withdrawing from the one or more adiabatic fixed bed reaction zones the converted-oxygenate product;
f) determining at an outlet of the one or more adiabatic fixed bed reaction zones an amount of one or more unconverted oxygenate compounds in the withdrawn converted-oxygenate product;
g) continuously adjusting the inlet temperature of the one or more feed streams in step b to maintain a constant amount of the one or more unconverted-oxygenate compounds as determined in step f of between 10 and 4000 ppmv.

2. Process according to claim 1, wherein the zeolite has a silica-to-alumina ratio (SAR) of at least 100.

3. Process according to claim 2, wherein the SAR is above 130.

4. Process according to claim 1, wherein said 1-D pore structure is ZSM-48, the pressure is at least 10 barg; the inlet temperature in step b of the one or more downstream adiabatic fixed bed reaction zones is 275° C. or higher, and the outlet temperature in step f of the one or more downstream adiabatic fixed bed reaction zones is 475° C. or lower; the SAR is at least 150, and the content of P in the conversion catalyst is 0.5-1.5 wt %.

5. Process according to claim 1, wherein the conversion catalyst is provided as an extrudate and wherein a P-precursor is impregnated in said extrudates for providing said content of P of 0.1-3 wt % or said 0.5-1.5 wt % in the conversion catalyst.

6. Process according to claim 1, wherein the one or more oxygenate compounds is methanol, dimethyl ether (DME), or combinations thereof.

7. Process according to claim 1, wherein in step d, said 05+ hydrocarbons are at least 75 wt % of said converted-oxygenate product.

8. Process according to claim 1, wherein: in step d, at least 40 wt % of said C5+ hydrocarbons are said hydrocarbons boiling in the jet fuel boiling range.

9. Process according to claim 8, wherein at least 40 wt % of said hydrocarbons boiling in the jet fuel range are olefins.

10. Process according to claim 8, wherein at least 4 wt % of said hydrocarbons boiling in the jet fuel range are C13+ hydrocarbons.

11. Process according to claim 1, wherein d the C5+ hydrocarbon fraction of the converted-oxygenate product, comprises C5-C7 hydrocarbons, and at least 40 wt % of said C5-C7 hydrocarbons are olefins.

12. Process according to claim 1, wherein in step g said constant amount of the one or more unconverted-oxygenate compounds is between 1000 and 3000 ppmv.

13. Process according to claim 1, wherein step g further comprises maintaining a constant level of conversion of the one or more oxygenate compounds of between 93 and 99.9%.

14. Process according to claim 1, wherein the composition of the converted-oxygenate product comprising C5+ hydrocarbons is: paraffins (P): 4-11 wt %, iso-paraffins (I): 5-30 wt %, olefins (O): 40-75 wt %, naphthenes (N): 6-15 wt %, aromatics (A): 4-20 wt %., in which the sum of P+I+O+N+A (PIONA) is 100 wt %; or wherein the olefin content of said C5+ hydrocarbons is at least 75 wt %.

15. Process according to claim 1,
wherein said one or more downstream adiabatic fixed bed reaction zones of said first set (R1) operate in parallel; and R1 operates as an oxygenate-to-olefins reaction zone;
wherein the process further comprises:
providing a second set (R2) of one or more adiabatic fixed bed reaction zones operating in parallel, in which R2 operates as an oligomerization reaction zone;
and wherein R1 and R2 are operated in parallel at least with respect to:
the one or more feed streams of one or more oxygenate compounds; and/or
a first C4− hydrocarbon fraction, as a first recycle stream, produced in downstream first separation unit; and/or
at least a portion of a naphtha intermediate product (13), as a second recycle stream, produced in downstream second separation unit.

16. Process according to claim 15, wherein R1 and R2 are operated at the same pressure.

17. Process according to claim 1, further comprising:
h) separating in first separation unit the converted-oxygenate product, into:
said first C4− hydrocarbon fraction;
converted oxygenate product comprising C5+ hydrocarbons of which at least 30 wt % are said hydrocarbons boiling in the jet fuel range;
a process condensate comprising water and unconverted oxygenates;
i) recycling at least a portion of said first C4− hydrocarbon fraction, as first recycle stream, to inlet of R1 and/or to inlet of R2.

18. Process according to claim 17, further comprising:
j) separating in a second separation unit the converted-oxygenate product comprising C5+ hydrocarbons of which at least 30 wt % are said hydrocarbons boiling in the jet fuel range, into:
an intermediate jet fuel product comprising said hydrocarbons boiling in the jet fuel boiling range;
said intermediate naphtha product comprising hydrocarbons boiling in the naphtha boiling range;
an intermediate diesel product comprising hydrocarbons boiling in the diesel boiling range;
k) recycling the at least a portion of said intermediate naphtha product, as said second recycle stream, to inlet of R2 and/or to inlet of R1.

19. Process according to claim 18, further comprising hydroprocessing any of said: intermediate jet fuel product, intermediate naphtha product, and intermediate diesel product into, respectively, a jet fuel product, a naphtha product, and a diesel product.

20. Process according to claim 18, in which step j further comprises:
separating a second C4− hydrocarbon fraction as an off-gas stream, and combining at least a portion of the off-gas stream with said first C4− hydrocarbon fraction to form part of said first recycle stream.

21. Process for the conversion of oxygenates to hydrocarbons boiling in the jet fuel boiling range, comprising the steps of continuously:
a) providing one or more feed streams of one or more oxygenate compounds;
b) heating the one or more feed streams to an inlet temperature of a first set (R1) of one or more downstream adiabatic fixed bed reaction zones;
c) introducing the one or more heated feed streams into an inlet of R1;
d) converting in the one or more adiabatic fixed bed reaction zones of R1 the one or more heated feed streams in the presence of a conversion catalyst comprising a zeolite with a framework having a 10-ring pore structure, said 10-ring pore structure being a unidimensional (1-D) pore structure, to a converted-oxygenate product of which at least 30 wt % are said hydrocarbons boiling in the jet fuel boiling range; wherein said 1-D pore structure is any of *MRE (ZSM-48), MTT (ZSM-23), TON (ZSM-22), or combinations thereof, and the content of phosphorous (P) in the conversion catalyst is 0.1-3 wt % (0.1-3 wt % P);
e) withdrawing from the one or more adiabatic fixed bed reaction zones the converted-oxygenate product;
f) determining at an outlet of the one or more adiabatic fixed bed reaction zones an amount of one or more unconverted oxygenate compounds in the withdrawn converted-oxygenate product;
g) continuously adjusting the inlet temperature of the one or more feed streams in step b to maintain a constant amount of the one or more unconverted-oxygenate compounds as determined in step f of between 10 and 4000 ppmv.

22. Process according to claim 5, wherein the P-precursor is phosphoric acid.

23. Process according to claim 5, wherein 50-60 wt % of the conversion catalyst is extruded with alumina.

24. Process according to claim 1, wherein: in step d, at least 40 wt % of said C5+ hydrocarbons are C8-C17 hydrocarbons.

25. Process according to claim 15, wherein the oxygenate-to-olefins reaction zone isa methanol-to-olefins (MTO) reaction zone.

26. Process according to claim 25, wherein the methanol-to-olefins reaction zone is a MTO reactor.

27. Process according to claim 15, wherein the oligomerization reaction zone is an oligomerization reactor (OLI reactor).

28. Process according to claim 18, wherein the second separation unit is a fractionation unit.

29. Process according to claim 19, wherein the hydroprocessing comprises hydrogenating and/or hydrocracking.
